(19) United States
(12) Patent Application Publication
(10) Pub. No.: US 2023/0372395 A1
(43) Pub. Date: Nov. 23, 2023

(54) CAR CELLS TARGETING AN INSERTED LIGAND

(71) Applicant: Massachusetts Institute of Technology, Cambridge, MA (US)

(72) Inventors: Darrell J. Irvine, Arlington, MA (US); Angela Q. Zhang, Cambridge, MA (US); Laura Shang-Bin Elizabeth Chen, Kirkland, WA (US); Alexander Hostetler, Cambridge, MA (US)

(73) Assignee: Massachusetts Institute of Technology, Cambridge, MA (US)

(21) Appl. No.: 18/123,112

(22) Filed: Mar. 17, 2023

Related U.S. Application Data
(60) Provisional application No. 63/343,804, filed on May 19, 2022.

(51) Int. Cl. A61K 35/17; A61K 47/54; A61K 47/68

(52) U.S. Cl. CPC A61K 35/17; A61K 47/543; A61K 47/6889; C07K 2317/622; C07K 2317/92; C07K 2319/74

(57) ABSTRACT

The present disclosure relates generally to technologies comprising engineered immune cells that express chimeric antigen receptors (CARs) that specifically bind to a membrane-inserting amphiphilic ligand. Also disclosed herein are methods and compositions for treating a tumor.

### CROSS-REFERENCE TO RELATED APPLICATIONS

This application claims priority to U.S. Provisional Application No. 63/343,804, filed May 19, 2022, the entire contents of which are incorporated herein by reference.

### GOVERNMENT LICENSING RIGHTS STATEMENT

This invention was made with government support under CA247632 and GM007753 awarded by the National Institutes of Health. The government has certain rights in the invention.

### BACKGROUND

The present invention relates generally to the field of immunotherapy, particularly to the use of engineered immune cells that recognize a ligand previously inserted into a tumor cell.

U.S. Pat. No. 9,233,125 discloses certain CAR cells which recognize certain cancer cells marked with a tag antibody.

There is a need for more effective targeting of tumor cells with engineered immune cells, particularly for solid tumors.

### SUMMARY

In one aspect, the present disclosure provides a method of treating a tumor, comprising introducing a membrane-inserting amphiphilic ligand into a tumor of a subject in need of treatment followed by administering an engineered immune cell expressing a CAR—that specifically binds to the amphiphilic ligand. In some embodiments, the membrane-inserting amphiphilic ligand is a fluorescein isothiocyanate lipid amphiphile ligand. In some embodiments, the membrane-inserting amphiphilic ligand comprises 1,2-distearoyl-sn-glycero-3-phosphoethanolamine-PEG-FITC. In some embodiments, the engineered immune cell expressing a CAR that specifically binds to the amphiphilic ligand is a fluorescein isothiocyanate (FITC) lipid amphiphile-specific engineered immune cell.

In some embodiments, the membrane-inserting amphiphilic ligand comprises a therapeutic compound conjugated to an amphiphilic poly(ethylene glycol)-lipid.

In some embodiments, the membrane-inserting amphiphilic ligand is introduced into the tumor by intratumoral injection. In some embodiments, administering the engineered immune cell expressing a CAR that specifically binds to the amphiphilic ligand comprises systemic infusion of the engineered immune cell into the subject. In some embodiments, the method further comprises introducing the membrane-inserting amphiphilic ligand into dendritic cells in lymph nodes of the subject by subcutaneous injection. In some embodiments, the frequency of intratumoral injection is equal to or less than about once every 6 days.

In some embodiments, the tumor of a subject in need of treatment is a solid tumor.

In some embodiments, the engineered immune cell expressing a CAR comprises a CD28 costimulatory domain.

In another aspect, the present disclosure provides an engineered immune cell expressing a CAR that specifically binds to a membrane-inserted amphiphilic ligand of a tumor cell. In some embodiments, the membrane-inserting amphiphilic ligand is a fluorescein isothiocyanate lipid amphiphile ligand. In some embodiments, the chimeric antigen receptor recognizes the amphiphilic ligand. In some embodiments, the chimeric antigen receptor comprises amphiphilic-ligand specific scFV. In some embodiments, the chimeric antigen receptor further comprises a CD28 costimulatory domain. In some embodiments, the fluorescein isothiocyanate lipid amphiphile ligand is 1,2-distearoyl-sn-glycero-3-phosphoethanolamine-PEG-FITC.

### BRIEF DESCRIPTION OF THE DRAWINGS

FIG. 1A-1H demonstrates Amph-FITC tags cancer cells for CAR T cell-mediated killing. FIG. 1A shows a schematic of amph-FITC structure, insertion into cancer cell membranes, and recognition by FITC CAR T cells. FIG. 1B-1C shows B16F10 murine melanoma cells that were incubated with amph-FITC at indicated concentrations for 30 min at 37° C. in PBS, stained with an anti-FITC antibody, and analyzed by flow cytometry. Shown are representative histograms of total FITC (FIG. 1B) and anti-FITC signals (FIG. 1C). FIG. 1D shows results of B16F10 cells that were incubated with amph-FITC conjugates of varying PEG molecular weight as in FIG. 1B at the indicated concentrations and then stained with anti-FITC for flow cytometry analysis. Shown are median fluorescence intensities (MFIs) of FITC (left) and anti-FITC (right) signals. FIG. 1E demonstrates kinetic analysis of anti-FITC MFI of B16F10 cultured in RPMI following tagging in 100 nM amph-FITC. FIG. 1F shows histograms of MC38 colon cancer cells labeled with titrated concentrations of amph-FITC to yield the same FITC fluorescence per cell (top). Cancer cells were subsequently cultured with 4m5.3-28z CAR T cells at a 1:1 effector-to-target (E:T) ratio. FIG. 1G-1H shows coculture of FITC-specific E2-m28z CAR T cells or control untransduced T cells with B16F10 (FIG. 1G) or CT2A (FIG. 111) cancer cells with or without coating by 100 nM amph-FITC at a range of E:T ratios. Shown are mean±standard deviation from triplicate samples. p values were determined by one-way ANOVA with Tukey's post hoc test. n.s., not significant; **p<0.01, ***p<0.001, ****p<0.0001.

FIG. 2A-2E demonstrates intratumoral administration of amph-FITC decorates cancer cells and draining lymph nodes with minimal labeling of other tissues. C57BL/6 mice were inoculated with 10⁶ B16F10 tumors in the flank, followed by intratumoral injection of 10 nmol DSPE-PEG₂ₖ-FITC when tumors reached 25 mm² in size. FIG. 2A shows confocal microscopy of B16F10 tumors 24 hours after intratumoral amph-FITC injection. One representative histological image from 2 tumors analyzed is shown. FIG. 2B shows results of 10⁶ B16F10 cells that were inoculated in C57BL/6 mice (n=4/group) and injected i.t. with 10 nmol amph-FITC when tumors were ˜25 mm² in size. 2 hours later, tumors were isolated with neighboring connective tissue, cryosectioned, and stained with anti-Trp1 antibody to identify melanoma cells, anti-FITC, and a cell membrane stain. Shown are representative sections from one PBS control and two amph-FITC-injected tumors. Scale bars are 200 μm. FIG. 2C shows biodistribution of amph-FITC 24 hours following intratumoral injection into B16F10 tumors (n=5 animals/group). FIG. 2D shows representative flow cytometry plots of amph-FITC-injected B16 tumors (gated on CD45− cells) stained with anti-FITC to detect surface-exposed antigen. FIG. 2E demonstrates immunophenotyping of B16F10 tumors in mice without prior lymphodepletion at 1, 24, and 48 hours following amph-FITC injection, quantifying the proportion of FITC+ anti-FITC+ double-positive cells (left), and density of FITC+ anti-FITC+ cells in the tumor (right) (n=5 animals/group). P values were determined by Mann-Whitney U test (FIG. 2D) or unpaired Student's t test (FIG. 2E). Shown are mean±standard deviation. n.s., not significant; *p<0.05, **p<0.01, ***p<0.001, ****p<0.0001.

FIG. 3A-3E demonstrates that FITC CAR T cells infiltrate into tumors and expand in the presence of amph-FITC. FIG. 3A shows a schematic and timeline of therapy in B16F10 tumors, including CAR T booster vaccine. FIG. 3B-3C shows localization of FLuc+ E2-m28z FITC CAR T cells in B16F10 tumor-bearing mice on day 8 (FIG. 3B) and quantification of whole mouse (top) and tumor (bottom) radiance (FIG. 3C) (n=5 animals/group). White dashed circle indicates location of flank tumor. FIG. 3D shows representative immunohistochemistry on CT2A tumors treated with i.t. amph-FITC ±amph-FITC vaccine, staining for CD8, at day 12 post-adoptive transfer. Scale bar is 50 μm. FIG. 3E shows quantification of CD8+ T cells per mm² per field of view (FOV) in 10 FOV per tumor, n=10 fields of view per condition. p values were determined by one-way ANOVA with Tukey's post hoc test. Error bars represent standard error of the mean (FIG. 3C) and standard deviation (FIG. 3E). n.s., not significant; ****p<0.0001.

FIG. 4A-4F demonstrates murine FITC CAR T cells combined with intratumoral amph-FITC have therapeutic activity in models of melanoma and glioma. FIG. 4A shows a schematic and timeline of therapy in CT2A tumor model. FIG. 4B shows tumor growth and overall survival of C57BL/6 mice (n=5 animals/group) bearing B16F10 tumors treated with E2-28z CAR T cells (as in FIG. 3A) with indicated combinations. FIG. 4C shows tumor growth of CT2A tumor-bearing C57BL/6 mice (n=5 animals/group) treated with amph-FITC, FITC CAR T cells, and vaccine composed of only CD8+ T cells or a combination of CD4+ and CD8+ T cells. FIG. 4D shows tumor growth and survival following treatment of CT2A tumor-bearing mice (n=5 animals/group) with CARs with low (E2.7), medium (E2), and high (4m5.3) affinities for FITC, including i.t. amph-FITC and vaccine. FIG. 4E-4F shows tumor growth (FIG. 4E) and survival (FIG. 4F) of CT2A tumor-bearing mice treated with CARs bearing CD28 or 4-1BB costimulatory domains across a range of binding affinities in combination with IT amph-FITC and amph-FITC vaccination (n=5 animals/group). Error bars represent standard error of the mean. p values were determined by two-way ANOVA (tumor growth curves) and log-rank (Mantel-Cox) test (survival curves). n.s., not significant; *p<0.05, **p<0.01, ***p<0.001.

FIG. 5A-5G demonstrates amph-FITC therapy induces epitope spreading to elicit an endogenous anti-tumor T cell response. FIG. 5A shows flow cytometry on treated CT2A tumors on day 34 after adoptive transfer of CD45.2+ FITC CAR T cells, quantifying CD4+ vs. CD8+ CD45.1+ tumor-infiltrating host T cells (n=4 animals/group). FIG. 5B shows CT2A tumor-bearing mice previously cured with CAR T and amph-FITC therapy were rechallenged vs. naïve mice with 10⁶ CT2A cancer cells on the opposite flank at day 92 following adoptive transfer (n=5 animals/group). FIG. 5C-5D shows representative flow plots of DCs (FIG. 5C) in tumors and TDLN of mice bearing tdTomato⁺ CT2A tumors at 12 days post-adoptive transfer and quantification (FIG. 5D) of tdTomato⁺ DCs in TDLN (n=3 animals/group for no CAR T group, n=4 for CAR T+i.t. FITC group). FIG. 5E-5F shows expression of activation markers CD86 (FIG. 5E) and CCR7 (FIG. 5F) in TDLN DCs at 12 days post-adoptive transfer with representative histograms (n=3 animals/group for no CAR T group, n=5 for CAR T+i.t. FITC group). FIG. 5G shows ELISPOT on spleens of CT2A tumor-bearing mice on day 37 post adoptive transfer (n=5 animals/group). Splenocytes were stimulated with irradiated CT2A cancer cells. P values were determined by unpaired Student's t test. Error bars represent standard deviation. n.s., not significant; *p<0.05, **p<0.01.

FIG. 6A-6E demonstrates amph-FITC CAR T cell therapy is efficacious when translated to human FITC CAR T cells in human solid tumor xenografts. FIG. 6A shows expression of humanized FITC CARs. FIG. 6B shows a schematic and timeline of therapy in NSG mice. FIG. 6C-6D shows bioluminescence imaging (FIG. 6C) and quantification (FIG. 6D) of FLuc⁺ CAR T cell trafficking in NSG mice (n=5 animals/group). FIG. 6E shows MSTO-211H tumor growth of NSG mice following treatment with E2-hBBz CAR T cells (n=10 animals/group). P values were determined by two-way ANOVA. Error bars represent standard error of the mean. **p<0.01, ***p<0.001.

FIG. 7A-7C shows characterization of kinetics of amph-FITC tagging. FIG. 7A-7B shows MC38 murine colon carcinoma cells (FIG. 7A) and CT-2A murine glioma cells (FIG. 7B) were incubated with amph-FITC at 100 nM for 30 min at 37° C. in PBS, stained with an anti-FITC antibody, and analyzed by flow cytometry. Shown are representative histograms of total FITC (left) and anti-FITC signals (right). FIG. 7C shows kinetic analysis of amph-FITC MFI of amph-FITC+ B16F10 cultured in RPMI following tagging.

FIG. 8A-8D demonstrates that murine T cells are effectively transduced to express FITC-specific CARs. FIG. 8A shows CAR expression in murine CD8⁺ 4m5.3-28z and E2-28z FITC CAR T cells, detected by staining of myc tag. FIG. 8B shows gating for exhaustion and memory phenotypes of FITC CAR T cells two days post-transduction. FIG. 8C shows CAR T cell phenotypes prior to adoptive transfer. (n=3 technical replicates). FIG. 8D shows mean PD-1/TIM-3 expression by CAR T cells prior to adoptive transfer. (n=3 technical replicates).

FIG. 9A-9C demonstrates biodistribution of intratumorally injected amph-FITC. FIG. 9A shows gating strategy for tumor immunophenotyping. FIG. 9B-9C show percent FITC+ anti-FITC+ double-positive cells (FIG. 9B) and density of FITC+ anti-FITC+ cells (FIG. 9C) in B16F10 tumors in C57BL/6 mice lymphodepleted with 5 Gy TBI at 1, 24, and 48 hours following amph-FITC injection (n=5 animals/group). P values were determined by unpaired Student's t test. Error bars represent standard deviation. *p<0.05, **p<0.01, ***p<0.001, ****p<0.0001.

FIG. 10 provides sample histograms of labeled cell types. Representative histograms of FITC and anti-FITC signal of cancer cells and immune cell subsets in lymphoreplete (left) and lymphodepleted (TBI, right) mice at 1, 24, and 48 hours following amph-FITC injection.

FIG. 11A-11C demonstrates that FITC CAR T cells expand in the peripheral blood and tumor with amph-FITC injection. FIG. 11A shows a timeline of therapy in MC38 tumor-bearing C57BL/6 mice. FIG. 11B-11C show CAR T cells in the peripheral blood (FIG. 11B, day 4) and tumor (FIG. 11C, day 11) (n=5 animals/group). P values were determined by unpaired Student's t test. Error bars represent standard deviation. *p<0.05, **p<0.01, ***p<0.001, ****p<0.0001.

FIG. 12A-12B demonstrates expression of CAR variants in murine CD8⁺ T cells. FIG. 12A-12B show histograms (FIG. 12A) and quantification (mean±SD) (FIG. 12B) of CAR expression 24 hours post-transduction of primary murine CD8⁺ T cells for FITC scFvs of varying affinity and with CD28 vs. 4-1BB costimulatory domains. P values were determined by unpaired Student's t test. Error bars represent standard deviation. ****p<0.0001.

FIG. 13A-13B demonstrates determination of FITC CAR T and amph-FITC therapy. FIG. 13A shows a comparison of tumor growth (left) and survival (right) in CT2A tumor-bearing mice treated with CAR T cells followed by intratumoral amph-FITC every 3 or 6 days (n=5 animals/group). FIG. 13B shows a comparison of tumor growth (left) and survival (right) in CT2A tumor-bearing mice treated with FITC CAR T cells prepared in mIL-2 or a combination of mIL-7 and mIL-15 (n=5 animals/group). P values were determined by two-way ANOVA (tumor growth curves) and log-rank (Mantel-Cox) test (survival curves). Error bars represent standard deviation. n.s., not significant; *p<0.05, **p<0.01, ***p<0.001, ****p<0.0001.

FIG. 14A-14D demonstrates FITC CAR T and amph-FITC therapy bears minimal toxicity. FIG. 14A shows a timeline of therapy in CT2A tumor-bearing C57BL/6 mice. FIG. 14B shows body weight of B16F10 tumor-bearing C57BL/6 mice over the course of therapy (n=5 animals/group). FIG. 14C-14D show quantification of serum cytokines (FIG. 14C, n=5 animals/group) and serum ALT and AST (FIG. 14D, n=7 animals/group) at 3 and 12 days post-adoptive transfer. P values were determined by two-way ANOVA (body weight curves) or unpaired Student's t test. Error bars represent standard error of the mean (FIG. 14B) and standard deviation (FIG. 14C). *p<0.05, **p<0.01, ***p<0.001, ****p<0.0001.

FIG. 15A-15F demonstrates expansion and tumor localization of human FITC CAR T cells in NSG mice. FIG. 15A shows in vitro cytotoxicity of FITC CAR T cells with amph-FITC⁺ MSTO-211H human mesothelioma cancer cells at 1:1 ET ratio. FIG. 15B shows comparison of luciferase signal from FLuc⁺ CAR T cells over time in mice treated with only CD8⁺ 4m5.3-h28z CAR T cells or a combination of CD4⁺ and CD8⁺ CAR T cells (n=5 animals/group). FIG. 15C-15D shows a gating strategy for immunophenotyping human CAR T cells (FIG. 15C) and phenotypic composition of CAR T cells recovered from spleens of NSG mice 23 days post-adoptive transfer (FIG. 15D, n=5 animals/group). FIG. 15E shows quantification of CD4⁺ and CD8⁺ CAR T cells in the spleen on day 40 post-adoptive transfer (n=5 animals/group). FIG. 15F shows whole mouse radiance over time as a surrogate of CAR T cell expansion (n=5 animals/group). Error bars represent standard error of the mean.

FIG. 16A-16F shows that local amph-FITC redirection of CAR T cells leads to a systemic anti-tumor immune response. FIG. 16A-16C shows C57BL/6 mice (untreated n=10, treated n=8) were inoculated with CT-2A tumor cells on opposite flanks, then treated with FITC-CAR T cells, amph-FITC vaccination, and i.t. amph-FITC only in the 1° lesion. Shown are the timeline and schematic of the treatment (FIG. 16A), mean tumor size (FIG. 16B), and overall survival (FIG. 16C) over time. FIG. 16D-16F shows C57BL/6 mice (n=10/group) were inoculated with B16F10 tumor cells on opposite flanks, then treated with FITC-CAR T cells, amph-FITC vaccination, and i.t. amph-FITC only in the 1° lesion. Shown are the timeline of the treatment (FIG. 16D), mean tumor size (FIG. 16E), and overall survival (FIG. 16F) over time. Error bars represent standard error of the mean. P values were determined by two-way ANOVA (tumor growth curves) and log-rank (Mantel-Cox) test (survival curves). Ns, not significant; **p<0.01; ***p<0.001, ****p<0.0001.

FIG. 17A-17B demonstrates evaluation of chemotherapy vs. irradiation-based lymphodepletion. FIG. 17A shows groups of C57BL/6 mice bearing B16F10 tumors (n=5 animals/group) were lymphodepleted with 250 mg/kg cyclophosphamide and 50 mg/kg fludarabine administered one day before adoptive cell transfer, followed by treatment with intratumoral amph-FITC and amph-FITC vaccine boosting as indicated. FIG. 17B shows comparison of tumor growth in mice left untreated (n=7) vs. animals receiving lymphodepleting regimen of 5 Gy TBI (n=8) seven days after being inoculated with 3×10⁶ CT-2A cells. p values were determined by two-way ANOVA. Error bars represent standard error of the mean. Ns, not significant; **p<0.01.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

In practicing the present technologies, many conventional techniques in molecular biology, protein biochemistry, cell biology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Chimeric antigen receptor (CAR) immune cell therapy, including, for example CAR-T cells, are effective in treating certain hematological malignancies, but translation to use in solid tumors has been challenging. To date, selecting an effective target antigen is hampered by heterogeneous tumor antigen expression and target antigen expression in healthy (e.g., non-tumor, non-cancerous) tissues.

The present disclosure is based, at least in part, on technologies to promote engineered immune cell mediated destruction of any tumor, including, for example, solid tumors. Such technologies provide a solution to off-target engineered immune cell activities as a result of heterogeneous tumor antigen expression and target antigen expression in healthy tissues. In some embodiments, technologies of the present disclosure comprise the discovery and use of membrane-inserting amphiphilic ligands and engineered immune cells, wherein engineered immune cells express a CAR that specifically bind to such amphiphilic ligands. The disclosure provides, among other things, methods of treating a tumor, comprising introducing a membrane-inserting amphiphilic ligand into a tumor of a subject in need of treatment followed by administering an engineered immune cell expressing a CAR that specifically binds to the amphiphilic ligand.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about,” when used to modify a numerical value, indicates that deviations of up to 10% above and below the numerical value remain within the intended meaning of the recited value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the term “antibody” generally refers to an antibody comprising two light chain polypeptides and two heavy chain polypeptides (unless the context in which this term is used suggests otherwise). Antibodies include different antibody isotypes including IgM, IgG, IgA, IgD, and IgE antibodies. The term “antibody” includes, without limitation, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, llama, dogs, cats, rabbits, guinea pigs, gerbils, hamsters, rats, and mice. The antibody can be a purified or a recombinant antibody

As used herein, the term “antibody fragment,” “antigen binding fragment,” or similar terms refer to a fragment of an antibody that retains the ability to bind to a target antigen. Such fragments include, without limitation, a single chain antibody, a single chain Fv fragment (scFv), a Fab fragment, a Fab′ fragment, and a F(ab′)2 fragment. This term also includes, e.g., single domain antibodies such as camelid single domain antibodies. See, e.g., Muyldermans et al. (2001) Trends Biochem Sci 26:230-235; Nuttall et al. (2000) Ciirr Pharm Biotech 1:253-263; Reichmann et al. (1999) J Immunol Meth 231:25-38; PCT application publication nos. WO 94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed. In addition, intrabodies, minibodies, triabodies, and diabodies are also included in the definition of antibody fragments and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12): 1121-1123; Rondon and Marasco (1997) Annual Review of Microbiology 5F257-283, the disclosures of each of which are incorporated herein by reference in their entirety.

As used herein, an “antigen” refers to a molecule to which an antibody, antibody fragment, and/or Chimeric Antigen Receptor can selectively bind. The target antigen may be a protein (e.g., an antigenic peptide), small molecule, carbohydrate, nucleic acid, lipid, hapten, or other naturally occurring or synthetic compound. An antigen may also be administered to an animal subject to generate an immune response in the subject.

As used herein, the term an “amino acid substitution” or “substituted” (when such term is referred to a substituted amino acid) refers to the replacement of at least one existing amino acid residue in a predetermined amino acid sequence with a different amino acid residue. The term “amino acid insertion” refers to the incorporation of at least one additional amino acid into a predetermined amino acid sequence. While the insertion will usually consist of the insertion of one or two amino acid residues, larger “peptide insertions,” can also be made. The replaced or inserted amino acid residue(s) may be naturally occurring or non-naturally occurring (modified).

The term “amino acid deletion” refers to the removal of at least one amino acid residue from a predetermined amino acid sequence.

By “binding affinity” is meant the strength of the total noncovalent interactions between a single binding site of a molecule (e.g., a CAR) and its binding partner (e.g., an antigen, a ligand). Without wishing to be bound by theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent).

As used herein, a “conservative amino acid substitution” is one in which the amino acid residue is replaced with an amino acid residue having a similar side chain. Families of amino acid residues having similar side chains have been defined in the art, including: (1) hydrophobic side chains: norleucine, Met, Ala, Val, Leu; (2) neutral hydrophilic side chains: Cys, Ser, Thr, Asn, Gin; (3) acidic side chains: Asp, Glu; (4) basic side chains: His, Lys, Arg; (5) side chains that influence chain orientation: Gly, Pro; and (6) aromatic side chains: Trp, Tyr, Phe. For example, a non-conservative amino acid substitution is a substitution of an amino acid residue with an amino acid residue with a substantially different side chain (i.e., an amino acid residue that is a member of a different family). In some embodiments, a conservative amino acid substitution is made by considering the hydropathic index of the amino acid residue. Each amino acid is assigned a hydropathic index on the basic of its hydrophobicity and charge characteristics. They are: lie (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5). The importance of the hydropathic amino acid index in conferring interactive function on a polypeptide is understood in the art (see, e.g., Kyte et al (1982) J Mol Biol 157:105-131). In some embodiments, a conservative amino acid substitution is made by replacing one amino acid residue with another amino acid residue having a the same or similar (e.g., within about +2, +1.5, +1, +0.5, −0.5, −1, −1.5, or −2) hydropathic index. In some embodiments, a conservative amino acid substitution is made by considering the hydrophilicity of the amino acid residue. The following hydrophilicity values have been assigned: Arg (+3.0); Lys (+3.0+1); Asp (+3.0+1); Glut (+0.2); Gly (0); Thr (−0.4); Pro (−0.5+1); Ala (−0.5); His (−0.5); Cys (−1.0); Met (−1.3); Val (−1.5); Leu (−1.8); lie (−1.8); Tyr (−2.3); Phe (−2.5); and Trp (−3.4). In some embodiments, a conservative amino acid substitution is made by replacing one amino acid residue with another amino acid residue having a the same or similar (e.g., within about +2, +1.5, +1, +0.5, −0.5, −1, −1.5, or −2) hydrophilicity. Exemplary amino acid substitutions are set forth in Table 2:

TABLE 2
Conservative amino acid substitutions

| Original | | Preferred |
|---|---|---|
| Residue | Exemplary Substitution | Substitution |
| Ala | Val, Leu, Ile | Val |
| Arg | Lys, Gln, Asn | Lys |
| Asn | Gln | Gln |
| Asp | Gln | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Nle | Leu |
| Leu | Nle, Ile, Val, Met, Ala, Phe | Ile |
| Lys | Arg, Dbu, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Nle | Leu |

Nle = norleucine
Dbu = 2,4-diaminobutyric acid

As used herein, the term, “co-stimulatory signaling domain,” or “costimulatory domain”, refers to the portion of the CAR comprising the intracellular domain of a co stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), 0X40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-IBB, other costimulatory domains are contemplated for use with the CARs described herein. The inclusion of one or more co-stimulatory signaling domains can enhance the efficacy and expansion of engineered immune cells expressing CAR receptors. The intracellular signaling and co-stimulatory signaling domains can be linked in any order in tandem to the carboxyl terminus of the transmembrane domain.

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the engineered immune cells administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. As used herein the term “KD” or “Kd” has the same meaning as commonly understood by one of ordinary skill in the art, and refers to the equilibrium dissociation constant of a binding reaction, for example, between an antibody (or antigen binding fragment thereof or CAR) and an antigen or ligand. The value of KD is a numeric representation of the ratio of the antibody off-rate constant (koff) to the antibody on-rate constant (kon). The value of KD is inversely related to the binding affinity of an antibody (or antigen binding fragment thereof or CAR) to an antigen or ligand. The smaller the KD value the greater the affinity of the antibody (or antigen binding fragment thereof or CAR) for its antigen or ligand. Affinity can be measured by any method known in the art.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “ligand” refers to a molecule that binds to a receptor (e.g., a CAR). In particular, the ligand binds a receptor on another cell, allowing for cell-to-cell recognition and/or interaction.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 amino acid residues.

As used herein, the term “percent (%) amino acid sequence identity” or “percent sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are known in the art, for instance, using publicly available computer software such as BFASTp, BFAST-2, AFIGN (e.g., AFIGN-2) or Megalign (DNASTAR) software. To obtain gapped alignments for comparison purposes, Gapped BEAST can be utilized as described in Altschul el ah, (1997) Nucleic Acids Res. 25(17):3389-3402. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, the terms “specific binding,” “specifically binds,” “selective binding,” and “selectively binds,” are intended to mean that an antibody or antigen-binding fragment thereof or a CAR that exhibits appreciable affinity for a particular antigen or ligand (e.g., a membrane-inserting amphiphilic ligand described herein) and, generally, does not bind to, or substantially does not bind to, other antigens or ligands. “Appreciable” or preferred binding includes binding with a KD of 10⁷, 10⁸, 10⁹, or 10¹⁰ M or better. The KD of an antibody, antigen-binding fragment or CAR—antigen or ligand interaction (the affinity constant) indicates the concentration of antibody, antigen-binding fragment, or CAR at which 50% of antibody, antigen-binding fragment, or CAR and antigen or ligand molecules are bound together. Thus, at a suitable fixed antigen concentration, 50% of a higher (i.e., stronger) affinity antibody, antigen-binding fragment, or CAR will bind antigen or ligand molecules at a lower antibody, antigen-binding fragment, or CAR concentration than would be required to achieve the same percent binding with a lower affinity antibody, antigen-binding fragment, or CAR. Thus a lower KD value indicates a higher (stronger) affinity. As used herein, “better” affinities are stronger affinities, and are of lower numeric value than their comparators, with a KD of 10⁷M being of lower numeric value and therefore representing a better affinity than a KD of 10⁶ M. Affinities better (i.e., with a lower KD value and therefore stronger) than 10⁷ M, preferably better than 10⁸ M, are generally preferred. Values intermediate to those set forth herein are also contemplated, and a preferred binding affinity can be indicated as a range of affinities.

As used herein, the term “T-cell” includes naive T cells, CD4⁺ T cells, CD8⁺ T cells, memory T cells, activated T cells, anergic T cells, tolerant T cells, chimeric B cells, and antigen-specific T cells.

As used herein, the term “vector” has the same meaning as commonly understood by one of ordinary skill in the art, and refers to a nucleic acid molecule capable of transporting another nucleic acid molecule to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors used in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Membrane-Inserting Amphiphilic Ligands

In one aspect, the present disclosure provides membrane-inserting amphiphilic ligands. Membrane-inserting amphiphilic ligands can insert their lipid tails into the plasma membrane of cells (e.g., target cells, including, for example tumor cells) in vitro and in vivo with minimal to no toxicity, leading to cell surface display of membrane-inserting amphiphilic ligands. In some embodiments, membrane-inserting amphiphilic ligands redirect engineered immune cells expressing a CAR that specifically bind to the amphiphilic ligand, as described herein, to the cells comprising the amphiphilic ligands on their cell surface. Such membrane-inserting amphiphilic ligands on the cell surface enable recognition by engineered immune cells bearing amphiphilic ligand-specific chimeric antigen receptors, thereby triggering engineered immune cell activation, proliferation, and killing of the cells comprising the amphiphilic ligands on their cell surface.

In some embodiments, a membrane-inserting amphiphilic ligand is or comprises a fluorescein isothiocyanate lipid amphiphile ligand. In some embodiments, a membrane-inserting amphiphilic ligand comprises 1,2-distearoyl-sn-glycero-3-phosphoethanolamine (DSPE)-PEG-FITC.

In some embodiments, membrane-inserting moieties (e.g., non-amphiphilic molecules) can be used. A plurality of membrane-inserting moieties are known in the art and one of ordinary skill would readily recognize and understand how to select and use such membrane-inserting moieties in accordance with the present disclosure.

In some embodiments, a membrane-inserting amphiphilic ligand comprises a amphiphilic poly(ethylene glycol) (PEG)-lipid. A plurality of amphiphilic PEG-lipids are known in the art and one of ordinary skill would readily recognize and understand how to select and use a known PEG-lipid in accordance with the present disclosure. PEG-lipids include, for example and without limitation, comprise dimyristoyl glycerol (DMG)-PEG 2000, Methoxypoly(ethylene glycol) (MPEG)2000C-DMG, α-[2-(ditetradecylamino)-2-oxoethyl]-ω-methoxy-poly(oxy-1,2-ethanediyl) (ALC-0159), 1,2-Dioleoyl-sn-glycero-3-phosphoethanolamine (DOPE)-PEG-Amine, molecular weight (MW) 1,000, DOPE-PEG-Amine, MW 2,000, DOPE-PEG-Amine, MW 3,400, DOPE-PEG-Amine, MW 5,000, DOPE-PEG-COOH, MW 1,000, DOPE-PEG-COOH, MW 2,000, DOPE-PEG-COOH, MW 3,400, DOPE-PEG-COOH, MW 5,000, DOPE-PEG-Mal, MW 1,000, DOPE-PEG-Mal, MW 2,000, DOPE-PEG-Mal, MW 3,400, DOPE-PEG-Mal, MW 5,000, DOPE-mPEG, MW 1,000, DOPE-mPEG, MW 2,000, DOPE-mPEG, MW 5,000, 1,2-Dimyristoyl-sn-Glycero-3-Phosphoethanolamine (DMPE)-mPEG, MW 2000, DSPE-PEG-NH2, MW 1,000, DSPE-PEG-Amine, MW 2,000, DSPE-PEG-Amine, MW 3,400, DSPE-PEG-Amine, MW 5,000, DSPE-PEG4-acid, DSPE-PEG-COOH, MW 1,000, DSPE-PEG-COOH, MW 3,400, DSPE-PEG-CH2COOH, MW 2,000, DSPE-PEG-CH2COOH, MW 5,000, DSPE-PEG-NHS, MW 600, DSPE-PEG-NHS, MW 1,000, DSPE-PEG-NHS, MW 2,000, DSPE-PEG-NHS, MW 3,400, DSPE-PEG-NHS, MW 5,000, DSPE-PEG13-TFP, DSPE-PEG5-azide, DSPE-PEG-Azide, MW 1,000, DSPE-PEG-Azide, MW 2,000, DSPE-PEG-Azide, MW 3,400, DSPE-PEG-Azide, MW 5,000, DSPE-PEG4-DBCO, DSPE-PEG-dibenzocycolctyne (DBCO), MW 2,000, DSPE-PEG-DBCO, MW 3,400, DSPE-PEG-DBCO, MW 5,000, DSPE-PEG5-propargyl, DSPE-PEG8-Mal, DSPE-PEG12-Mal, DSPE-PEG-Maleimide, MW 2,000, DSPE-PEG-Maleimide, MW 5,000, DSPE-PEG-Ald, MW 1,000, DSPE-PEG-Ald, MW 2,000, DSPE-PEG-Ald, MW 3,400, DSPE-PEG-Ald, MW 5,000, DSPE-PEG-Biotin, MW 2,000, DSPE-PEG-Biotin, MW 3,400, DSPE-PEG-Folate, MW 1,000, DSPE-PEG-Folate, MW 2,000, DSPE-PEG-Folate, MW 3,400, DSPE-PEG-Folate, MW 5,000, DSPE-PEG-OH, MW 1,000, DSPE-PEG-OH, MW 2,000, DSPE-PEG-OH, MW 3,400, DSPE-PEG-OH, MW 5,000, DSPE-PEG-SH, MW 1,000, DSPE-PEG-SH, MW 2,000, DSPE-PEG-SH, MW 3,400, DSPE-PEG-SH, MW 5,000, DSPE-PEG-SPDP, MW 1,000, DSPE-PEG-SPDP, MW 3,400, DSPE-PEG-SPDP, MW 5,000, DSPE-PEG-2-Aminoethyl-alpha-Mannopyranoside, MW 2,000, DSPE-PEG-IA, MW 2,000, DSPE-PEG-IA, MW 3,400, DSPE-PEG-IA, MW 5,000, DSPE-PEG-Boronate, MW 2,000, DSPE-PEG-Vinylsulfone, MW 1,000, DSPE-PEG-Vinylsulfone, MW 2,000, DSPE-PEG-Vinylsulfone, MW 3,400, DSPE-PEG-Vinylsulfone, MW 5,000, DSPE-PEG-Cy3, MW 2,000, DSPE-PEG-Cy3, MW 3,400, DSPE-PEG-Cy3, MW 5,000, DSPE-PEG-Cy5, MW 3,400, DSPE-PEG-Cy5, MW 5,000, DSPE-PEG-FITC, MW 1,000, DSPE-PEG-FITC, MW 2,000, DSPE-PEG-FITC, MW 3,400, DSPE-PEG-FITC, MW 5,000, DSPE-PEG-Rhodamine, MW 1,000, DSPE-PEG-Rhodamine, MW 2,000, DSPE-PEG-Rhodamine, MW 3,400, DSPE-PEG-Rhodamine, MW 5,000, m-PEG8-DSPE, m-PEG12-DSPE, m-PEG-DSPE, MW 550, m-PEG-DSPE, MW 1,000, m-PEG-DSPE, MW 2,000, m-PEG-DSPE, MW 3,000, m-PEG-DSPE, MW 5,000, Stearic acid-PEG-NHS, MW 1,000, Stearic acid-PEG-NHS, MW 2,000, Stearic acid-PEG-NHS, MW 3,400, Stearic acid-PEG-NHS, MW 5,000, Stearic acid-mPEG, MW 1,000, Stearic acid-mPEG, MW 2,000, Stearic acid-mPEG, MW 5,000, Stearic acid-PEG-Rhodamine, MW 1,000, Stearic acid-PEG-Rhodamine, MW 2,000, Stearic acid-PEG-Rhodamine, MW 3,400, Stearic acid-PEG-Rhodamine, MW 5,000, Stearic acid-PEG-FITC, MW 1,000, Stearic acid-PEG-FITC, MW 2,000, Stearic acid-PEG-FITC, MW 3,400, Stearic acid-PEG-FITC, MW 5,000, (R)-2,6-Bis-(m-PEG4)-amidohexanoic acid, (S)-2,6-Bis-(m-PEG8)-amidohexanoic acid, Pentacosadiynoic acid-MPEG, MW 1,000, Pentacosadiynoic acid-MPEG, MW 2,000, Pentacosadiynoic acid-MPEG, MW 5,000.

In some embodiments, a membrane-inserting amphiphilic ligand comprises a therapeutic compound conjugated to an amphiphilic poly(ethylene glycol)-lipid. In some embodiments, a therapeutic compound comprises one or more small molecules, polypeptides, polysaccharides and/or synthetic polymers. In some embodiments, a therapeutic compound is any small molecule, polypeptide, polysaccharide and/or synthetic polymer that can be conjugated to an amphiphilic poly(ethylene glycol)-lipid and targeted by an engineered immune cell described herein.

In some embodiments, a therapeutic compound is or comprises a small molecule, including, for example and without limitation, FITC, Texas Red, BODIPY, Cy3, Camptothecin, Doxorubicin, and Irinotecan, or derivatives thereof.

In some embodiments, a therapeutic compound is or comprises a polypeptide, such as, for example, enzymes, monoclonal antibodies, and/or cytokines or fragments thereof. Non-limiting examples of polypeptides include, arginine deaminase, asparaginase, blue fluorescent protein, epidermal growth factor, granulocyte-colony stimulating factor, green fluorescent protein, interferons (e.g., interferon-α, interferon-β, etc.), mCherry, programmed cell death protein 1, programmed death ligand 1, red fluorescent protein, vascular endothelial growth factor receptors, yellow fluorescent protein, etc.

### Chimeric Antigen Receptor (CAR)

In one aspect, the present disclosure provides Chimeric Antigen Receptors (CARs) and nucleic acids encoding CARs. CARs are genetically-engineered, artificial membrane-bound proteins that, when expressed on an engineered immune cell (e.g., a T cell), direct such an engineered immune cell to a specific target molecule (e.g., ligand, antigen), and generally stimulates the engineered immune cell to kill the cell displaying the specific target molecule (e.g., a target cell, including for example, a tumor cell). Thus, CARs can be used to impart a desired target specificity to engineered immune cells, such as specificity to a tumor comprising a membrane-inserting amphiphilic ligand as described herein. In some embodiments, transfer of the coding sequence of the CAR is facilitated by nucleic acid vector, such as a retroviral vector. A plurality of appropriate technologies for transfer of coding sequences will be readily apparent to one of ordinary skill in the art.

In some embodiments, a CAR comprises three domains: (i) an extracellular domain typically comprising a signal peptide, a ligand, and/or an antigen recognition region (e.g., scFv), and a flexible spacer; (ii) a transmembrane domain; and (iii) an intracellular domain typically comprising one or more signaling domains. In some embodiments, a CAR can further comprise a linker.

In some embodiments, a subject CAR of the present disclosure may comprise any combination of any of the domains described herein (e.g., extracellular domains, transmembrane domains, costimulatory domains, intracellular domains, and/or hinge domains).

The target molecule specificity of a CAR is imparted by the extracellular domain of the CAR. In some embodiments, engagement of a CAR extracellular domain (e.g., a ligand and/or antigen recognition region, including, for example, a scFv) with its specific target molecule results in clustering of the CAR and delivers an activation stimulus to the CAR-containing cell. In some embodiments, CARs redirect engineered immune cells (e.g., T cells) to a particular target cell comprising a CAR-specific target molecule (e.g., a membrane-inserting amphiphilic ligand as described herein). Upon binding of the CAR to a specific target molecule, downstream signaling initiates, for example, production of molecules that can mediate cell death of a target cell (e.g., a tumor cell comprising a membrane-inserting amphiphilic ligand).

Extracellular Domains

A CAR extracellular domain (e.g., binding domain) comprises a ligand and/or antigen recognition region and is the region of a CAR that imparts specificity for binding a specific target molecule (e.g., an antigen, a ligand). In some embodiments, a specific target molecule is or comprises, for example, a polypeptide, a carbohydrate, a glycolipid, and/or a small molecule, including, for example, membrane-inserting amphiphilic ligands described herein. In some embodiments, a CAR comprises affinity to a specific target molecule (e.g., a membrane-inserting amphiphilic ligand described herein) on a target cell (e.g., a tumor cell). The specific target molecule may include, for example, any type of protein, small molecule (e.g., membrane-inserting amphiphilic ligands described herein), or epitope associated with the target cell. For example, in some embodiments, a CAR may comprise affinity to any membrane-inserting amphiphilic ligand described herein.

The extracellular domain can include any region that binds to a specific target molecule (e.g., a ligand, an antigen). In some embodiments, the extracellular domain comprises or is derived from natural binding proteins or receptors. In some embodiments, the extracellular domain comprises or is derived from an antigen binding fragment (Fab) or a single-chain variable fragment (scFv). In some embodiments, a scFv is specific to a membrane-inserting amphiphilic ligand as described herein. Non-limiting examples of a ScFv include those comprising, or derived from, 4m5.3 (see, e.g., Boder, E. T et al. Proceedings of the National Academy of Sciences of the United States of America 97, 10701-10705 (2000)), E2 (see, e.g., Vaughn, T. J. et al., Nature Medicine 2, 534-539 (1996)), or E2 with a His-H58-Ala mutation (“E2.7”) (see, e.g., Pedrazzi, G. et al., FEBS Letters 415, 289-293 (1997)).

In some embodiments, a CAR of the present disclosure may have affinity for one or more specific target molecules (e.g., ligands, antigens) on one or more target cells (e.g., tumor cells). In some embodiments, a CAR may have affinity for one or more specific target molecules on a single target cell. In some such embodiments, a CAR is a bispecific CAR or a multispecific CAR. In some embodiments, a CAR comprises one or more target molecule-specific recognition regions that confer affinity for one or more specific target molecules. In some embodiments, a CAR comprises one or more target-specific recognition regions that confer affinity for the same specific target molecule. For example, a CAR comprising one or more target-specific recognition regions having affinity for the same specific target molecule could bind distinct epitopes of the specific target molecule. When a plurality of target-specific recognition regions are present in a CAR, the recognition regions may be arranged in tandem and may be separated by linker peptides. For example, in a CAR comprising two target-specific recognition regions, the recognition regions are connected to each other covalently on a single polypeptide chain, through a polypeptide linker, an Fc hinge region, or a membrane hinge region.

In some embodiments, a polypeptide linker is rich in glycine. Without wishing to be bound by any one theory, a glycine-rich linker is utilized due to its enhanced solubility. In some embodiments, a polypeptide linker comprises serine and/or threonine. In some embodiments, a polypeptide linker links the heavy chain variable region and the light chain variable region of the extracellular domain. Non-limiting examples of linkers are disclosed in, for example, Shen et al, Anal. Chem. 80(6): 1910-1917 (2008) and WO 2014/087010, the contents of which are hereby incorporated by reference in their entireties. Those of skill in the art would readily understand appropriate polypeptide linkers for use in the present invention.

In some embodiments, an extracellular domain comprises a signal peptide. A variety of signal peptides are known in the art and those of ordinary skill in the art would be able to select an appropriate signal peptide for use in accordance with the present disclosure. In some embodiments, a signal peptide comprises a CD8α signal peptide. In some embodiments, a CD8α signal peptide comprises the amino acid sequence: MALPVTALLLPLALLLHAARP. In some embodiments, a signal peptide comprises a CD8α signal peptide (e.g., MALPVTALLLPLALLLHAARP) followed by a Myc-tag (EQKLISEEDL).

In certain embodiments, the extracellular domain of a presently disclosed CAR has a high binding specificity and high binding affinity to a membrane-inserting amphiphilic ligand as described herein. For example, in some embodiments, the extracellular domain of the CAR (for example, a scFv) binds to a particular tumor antigen with a dissociation constant (Kd) of about 1×10⁻⁵ M or less. In certain embodiments, the Kd is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, about 1×10⁻¹² M or less, about 5×10⁻¹³ M or less, about 1×10⁻¹³ M or less, about 5×10⁻¹⁴ M or less, about 1×10⁻¹⁴ M or less, about 5×10⁻¹⁵ M or less, about 4×10⁻¹⁵ or less, about 3×10⁻¹⁵ or less, about 2×10⁻¹⁵ or less, or about 1×10⁻¹⁵ M or less.

Binding of the extracellular domain of a presently disclosed CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular domain of the tumor antigen-targeted CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

In some embodiments, the extracellular domain of a CAR specifically binds to a membrane-inserting amphiphilic ligand described herein. In some embodiments, the extracellular domain of a CAR specifically binds to a membrane-inserted amphiphilic ligand present on the surface of a target cell (e.g., a tumor cell).

Extracellular domains described herein can be combined with any transmembrane domains described herein, any costimulatory domains described herein, any intracellular domains, and/or any hinge domains described herein.

Transmembrane Domains

A CAR transmembrane domain is a region that is capable of spanning at least a portion of the plasma membrane of a cell (e.g., an engineered immune cell). Different transmembrane domains can result in different receptor stability. In some embodiments, after specific target molecule recognition, receptors cluster and a signal is transmitted to the cell.

In some embodiments, a transmembrane domain is naturally associated with one or more of the domains in the CAR. In some embodiments, a CAR transmembrane domain can be designed to connect the extracellular domain of the CAR to the intracellular domain of the CAR. In some embodiments, a transmembrane domain can be selected or modified by amino acid substitution to avoid binding of such domains to the transmembrane domains of the same or different surface membrane proteins to minimize interactions with other members of the receptor complex.

In some embodiments, a transmembrane domain is derived from a natural source. In some embodiments, a transmembrane domain is derived from a synthetic source. Where the source is natural, a transmembrane domain may be derived from any membrane-bound or transmembrane protein, e.g., a Type I transmembrane protein. Where the source is synthetic, a transmembrane domain may be any artificial sequence that facilitates insertion of the CAR into a cell membrane, e.g., an artificial hydrophobic sequence. Examples of transmembrane domains include, without limitation, transmembrane domains derived from (i.e., comprise at least the transmembrane region(s) or a fragment thereof of) the alpha, beta or zeta chain of the T-cell receptor, CD28, CD2, CD3 epsilon, CD45, CD4, CD5, CD7, CD8 (e.g., CD8α), CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9.

In some embodiments, a transmembrane domain further comprises a hinge domain (also referred to as a spacer domain). A CAR hinge domain is a hydrophilic region located between the extracellular domain and the transmembrane domain. In some embodiments, a hinge domain facilitates proper protein folding of a CAR as described herein. In some embodiments, a hinge domain comprises a domain selected from Fc fragments of antibodies, hinge regions of antibodies, CH2 regions of antibodies, CH3 regions of antibodies, artificial hinge sequences or combinations thereof. In some embodiments, the hinge domain is an immunoglobulin heavy chain hinge region. In some embodiments, the hinge domain is a hinge domain polypeptide derived from a receptor (e.g., a CD8-derived hinge domain, including, for example CD8α). In some embodiments, artificial hinges made of polypeptides which may be as small as, three glycines (Gly), and/or CHI and CH3 domains of IgGs (such as human IgG4).

In some embodiments, a CAR of the present disclosure comprises a hinge domain that connects the extracellular domain with the transmembrane domain, which, in turn, connects to the intracellular domain. In some embodiments, a hinge domain is capable of supporting the extracellular domain recognize and bind to a specific target molecule on the target cells (see, e.g., Hudecek et al., Cancer Immunol. Res. (2015) 3(2): 125-135). In some embodiments, a hinge domain is a flexible domain, thus allowing the extracellular domain to have a structure to optimally recognize the specific structure and density of the specific target molecules on a target cell, such as tumor cell. Without wishing to be bound by any one theory, the flexibility of the hinge domain allows the hinge domain to adopt many different conformations.

In some embodiments, a transmembrane domain comprises a CD8α transmembrane domain. In some embodiments, a CAR comprises a CD8α transmembrane domain and a CD8α hinge domain.

Transmembrane domains described herein can be combined with any extracellular domains described herein, any costimulatory domains described herein, any intracellular domains, and/or any hinge domains described herein.

Intracellular Domains

A CAR intracellular domain is responsible for activation of at least one of the effector functions of the cell in which the CAR is expressed (e.g., an engineered immune cell). The intracellular domain transduces effector function signal and directs the cell (e.g., an engineered immune cell) to perform its function (e.g., destruction of a target cell). Examples of an intracellular domain for use in accordance with the present disclosure include, but are not limited to, the cytoplasmic portion of a surface receptor, co-stimulatory molecule, and/or any molecule that acts in concert to initiate signal transduction in the engineered immune cell, as well as any derivative or variant of these elements and any synthetic sequence that has the same functional capability.

For example, in some embodiments, an intracellular signaling domain includes, without limitation, the z chain of the T cell receptor complex or any of its homologs, e.g., h chain, FcsRFy and b chains, MB 1 (Iga) chain, B29 (Ig) chain, etc., human CD3 zeta chain, CD3 polypeptides (D, d and e), syk family tyrosine kinases (Syk, ZAP 70, etc.), src family tyrosine kinases (Lck, Fyn, Lyn, etc.), and other molecules involved in T cell transduction, such as CD2, CD5 and CD28. In some embodiments, an intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP 12, T cell receptor (TCR), CD8, CD27, CD28, 4-1BB (CD137), OX9, 0X40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD 127, CD160, CD19, CD4, CD8alpha, CD8beta, IL2Rbeta, IL2R gamma, IL7R alpha, ITGA4, VLA1, CD49a, ITGA4, IA4, CD49D, ITGA6, VLA-6, CD49f, IT gAD, CDlId, ITGAE, CD103, ITgAL, CDlla, LFA-1, ITGAM, CD lib, ITGAX, CDllc, ITGB1, CD29, ITGB2, CD 18, LFA-1, ITGB7, TNFR2, TRAN CE/R ANKL, DNAM1 (CD226), SLAMF4 (CD244, 2B4), CD84, CD96 (Tactile), CEACAMI, CRT AM, Ly9 (CD229), CD 160 (BY55), PSGL1, CD 100 (SEMA4D), CD69, SLAMF6 (NTB-A, Lyl08), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAPIO, and CD3z.

In some embodiments, the intracellular domain comprises a costimulatory signaling domain. In some embodiments, the intracellular signaling domain comprises an intracellular signaling domain. In certain embodiments, an intracellular domain comprises a costimulatory domain and an intracellular signaling domain. In certain embodiments, an intracellular domain comprises 4-1BB, CD28, and/or CD3ζ domains.

In some embodiments, an intracellular domain of a CAR comprises a costimulatory signaling domain which includes any portion of one or more costimulatory molecules, such as at least one signaling domain from CD2, CD3 (including, e.g., CD3ζ), CD8, CD27, CD28, 0X40, ICOS, 4-1BB, PD-1, any derivative or variant thereof, any synthetic sequence thereof that has the same functional capability, and any combination thereof.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by specific target molecule).

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

Intracellular domains described herein can be combined with any extracellular domains described herein, any costimulatory domains described herein, any transmembrane domains, and/or any hinge domains described herein.

### Engineered Immune Cells

In one aspect, the present disclosure provides engineered immune cells expressing any chimeric antigen receptor (CAR) described herein. In certain embodiments, provided herein are engineered immune cells transformed with a nucleic acid encoding any CAR described herein.

The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

T cells that can be for example, all types of immune cells expressing CD3 including T-helper cells (CD4⁺ cells), cytotoxic T-cells (CD8⁺ cells), natural killer T-cells, T-regulatory cells (Treg) and gamma-delta T cells.

In some embodiments, engineered immune cells of the presently disclosed subject matter can be macrophages. Macrophages are effector cells of the innate immune system that phagocytose bacteria and secrete both pro-inflammatory and antimicrobial mediators as well as play an important role in eliminating diseased and/or damaged cells via programmed cell death.

In some embodiments, an engineered immune cell provided herein is autologous to a subject to whom they are to be administered (e.g., after their modification to express a CAR as described herein). In certain embodiments, an engineered immune cell provided herein is allogeneic to a subject to whom they are to be administered (e.g., after their modification to express a CAR as described herein). In some embodiments, wherein allogeneic immune cells are used to prepare an engineered immune cell expressing a CAR, engineered immune cells can be selected that will decrease the possibility of graft-versus-host disease in a subject in need of treatment. In some embodiments, wherein allogeneic immune cells are used to prepare an engineered immune cell expressing a CAR, engineered immune cells can be co-administered with one or more immunosuppressive agents. In some embodiments, an engineered immune cell is obtained from a subject, optionally expanded, and transformed with a polynucleotide comprising a nucleic acid sequence encoding a CAR described herein, and optionally further expanded.

In some embodiments, immune cells provided herein have been isolated from, or expanded from a buffy coat (e.g., a fraction of an anticoagulated blood sample). In certain embodiments, a buffy coat has been obtained and/or prepared from a donor (e.g., a human donor). In certain embodiments, the donor is a healthy human donor. In certain embodiments, the donor is a human donor that has cancer.

In some embodiments, immune cells provided herein have been isolated from, or expanded from whole blood. In certain embodiments, whole blood has been obtained and/or prepared from a donor (e.g., a human donor). In certain embodiments, the donor is a healthy human donor. In certain embodiments, the donor is a human donor that has cancer.

In some aspects, immune cells are obtained from a subject with a disease and/or a condition. In some embodiments, the disease and/or condition is a tumor. In some embodiments, the disease and/or condition is a cancer.

In some aspects, immune cells are obtained from a subject with a disease and/or condition and genetically modified (e.g., in vitro) to express at least one CAR with specificity for at least one membrane-inserting amphiphilic ligand as described herein (e.g., a fluorescein isothiocyanate lipid amphiphile ligand). In some embodiments, an immune cell is genetically modified to express a CAR with specificity for a membrane-inserting amphiphilic ligand described herein is then administered to treat a tumor and/or cancer in a subject in need of treatment. In some embodiments, an engineered immune cell performs at least one effector function (e.g., immune-mediated cell death) that is stimulated or induced by the specific binding of the CAR to the membrane-inserting amphiphilic ligand and that is useful for treatment of a subject in need of treatment. In some embodiments, an engineered immune cell exerts its effector function (e.g., a cytotoxic T cell response) on a target cell (e.g., a tumor cell) when brought in contact or proximity to the target and/or the target cell (see, e.g., Chang and Chen (2017) Trends Mol Med 23(5):430-450).

Stimulation of an engineered immune cell (e.g., by binding of the extracellular domain of the CAR to a membrane-inserting amphiphilic ligand as described herein) can result in the activation of one or more anti-cancer activities of the engineered immune cell. For example, in some embodiments, stimulation of an engineered immune cell can result in an increase in cytotoxic activity or helper activity of an engineered immune cell.

In some embodiments, a subject's immune cells are genetically modified with a CAR (see, e.g., Sadelain et al, Cancer Discov. 3:388-398, 2013). For example, in some embodiments, an immune cell obtained from a subject in need of treatment is provided a recombinant nucleic acid encoding a CAR to generate an engineered immune cell. In some embodiments, an immune cell not obtained from a subject in need of treatment is provided a recombinant nucleic acid encoding a CAR to generate an engineered immune cell. In some embodiments, immune cells are allogenic cells that have been engineered to be utilized as an “off the shelf” adoptive cell therapy.

A plurality of methods are known in the art to introduce nucleic acids into cells (e.g., immune cells). Any suitable method known in the art to introduce any nucleic acids encoding a CAR as described herein into an immune cell can be used. Non-limiting examples of methods for introducing nucleic acid into an immune cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce CAR nucleic acids into immune cells and/or to introduce other genetic modifications into immune cells to enhance engineered immune cell activity (for use of CRISPR/Cas9 technology in connection with engineered immune cells, see e.g., U.S. Pat. Nos. 9,890,393; 9,855,297; US 2017/0175128; US 2016/0184362; US 2016/0272999; WO 2015/161276; WO 2014/191128; CN 106755088; CN 106591363; CN 106480097; CN 106399375; CN 104894068).

In some aspects, a method for producing an engineered immune cell described herein comprises: (i) obtaining cells from peripheral blood, cord blood or lymph (e.g., from peripheral blood mononuclear cells (PMBC)), (ii) optionally, purifying the obtained cells, (iii) optionally, expanding the cells, (iv) optionally, activating the cells (e.g., with an anti-CD3 antibody or an antigen binding fragment thereof and/or an anti-CD28 antibody or an antigen-binding fragment thereof), (v) optionally, expanding the activated cells, (vi) transducing the cells with an expression vector comprising a CAR described herein, (vii) isolating the cells expressing the CAR, and (viii) optionally, expanding the isolated cells.

In some aspects, a method for producing an engineered immune cell described herein comprises: (i) obtaining a pluripotent stem cell (iPSC) (ii) inducing iPSC to differentiate into a particular immune cell (such as CD8⁺ T cell), (iii) optionally, expanding the cells, (iv) transducing the cells with an expression vector comprising a CAR described herein, (v) isolating the cells expressing the CAR, and (vi) optionally, expanding the isolated cells.

In some aspects, a method for producing an engineered immune cell described herein comprises: (i) obtaining whole blood or a buffy coat (e.g., from a healthy donor), (ii) isolating human primary immune cells (e.g., T cells) from the whole blood or buffy coat, (iii) optionally, expanding the cells, (iv) transducing the cells with an expression vector comprising a CAR described herein, (v) isolating the cells expressing the CAR, and (vi) optionally expanding the isolated cells.

### Compositions

In one aspect, the present disclosure provides compositions (e.g., pharmaceutical compositions) comprising a membrane-inserting amphiphilic ligand as described herein. In some embodiments, membrane-inserting amphiphilic ligands as described herein of pharmaceutical compositions can be purified (e.g., separated from one or more undesired components).

In one aspect, the present disclosure provides compositions (e.g., pharmaceutical compositions) comprising engineered immune cells as described herein. In some embodiments, engineered immune cells as described herein of pharmaceutical compositions can be purified (e.g., separated from one or more undesired components).

In some embodiments, a pharmaceutical composition comprises a pharmaceutically acceptable carrier. Appropriate pharmaceutically acceptable carriers, including but not limited to excipients and stabilizers, are known in the art (see, e.g., Remington's Pharmaceutical Sciences (1990) Mack Publishing Co., Easton, PA).

In some embodiments, a pharmaceutical composition is a sterile composition. In some such embodiments, a pharmaceutical composition (e.g., a sterile composition) can comprise cells, tethering means (e.g., lipid nanoparticles) and/or proteins or polypeptides, preferably in a pharmaceutically-acceptable carrier (e.g., one or more compatible solid or liquid filler, diluents or encapsulating substances that are suitable for administration to a human or other subject contemplated herein). In some embodiments, the carrier can be an organic or inorganic ingredient, natural or synthetic, with which the cells, tethering means (e.g., lipid nanoparticles) and/or proteins or peptides are combined to facilitate administration. The components of the pharmaceutical compositions are commingled in a manner that precludes interaction that would substantially impair their desired pharmaceutical efficiency.

Pharmaceutically acceptable carriers may include, for example and without limitation, a buffer, an emulsifying agent, a suspending agent, a dispersing agent, an isotonic agent, a wetting agent, a chelating agent, a sequestering agent, a pH buffering agent, a solubility enhance, an antimicrobial agent, an anesthetic, and/or an antioxidant.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspending or dispersing agents, sweeteners, and waters of hydration. Exemplary excipients include, but are not limited to: saline, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, sucrose, dextrose, magnesium stearate, malt, maltitol, mannitol, methionine, methylcellulose, methyl paraben, microcrystalline cellulose, polyethylene glycol, glycerol, ethanol, polyvinyl pyrrolidone, povidone, starch (e.g., pregelatinized starch), propylene, propyl paraben, retinyl palmitate, shellac, silica gel, silicon dioxide, sodium carboxymethyl cellulose, sodium citrate, sodium stearate, sodium starch glycolate, sorbitol, starch (corn), stearic acid, talc, base cream, titanium dioxide, vitamin A, vitamin E, vitamin C, and xylitol.

In some embodiments, the pharmaceutical compositions disclosed herein may include at least one pharmaceutically acceptable salt. Examples of pharmaceutically acceptable salts that may be included in a composition of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like

In some embodiments, pharmaceutical compositions can be formulated such that they are suitable for administration to a subject (e.g., a human) in need of treatment. A pharmaceutical composition may be formulated for any route of administration.

In some embodiments, pharmaceutical compositions, when it is desirable to deliver them systemically, may be formulated for parenteral administration by injection, e.g., by bolus injection or continuous infusion. Such formulations can be prepared as liquid solutions, suspensions, emulsions or solid forms suitable making into a solution or suspension prior to injection. Formulations for injection may be presented in unit dosage form, e.g., in ampoules or in multi-dose containers. In some embodiments, pharmaceutical parenteral formulations include aqueous solutions of the ingredients. Aqueous injection suspensions may contain, for example, substances which increase the viscosity of the suspension, such as sodium carboxymethyl cellulose, sorbitol, or dextran. Alternatively, suspensions of ingredients may be prepared as oil-based suspensions. Suitable lipophilic solvents or vehicles include, for example, fatty oils such as sesame oil, or synthetic fatty acid esters, such as ethyl oleate or triglycerides, or liposomes. If administered parenterally, suitable pharmaceutically acceptable carriers may include, without limitation, physiological saline or phosphate buffered saline (PBS), or solutions containing, e.g., polyethylene glycol, polypropylene glycol or glucose.

A membrane-inserting amphiphilic ligand described herein and/or engineered immune cell described herein can be used or present in a therapeutically effective amount in the pharmaceutical composition disclosed herein. The therapeutically effective amount can be determined by standard clinical techniques.

The pharmaceutically acceptable compositions contemplated herein may include, in addition to a membrane-inserted amphiphilic ligand described herein and/or engineered immune cell described herein, an additional anti-cancer agent (e.g., any one, two, three or more anti-cancer agents described herein).

### Therapeutic Methods and Uses

In one aspect, the present disclosure provides technologies for treating a tumor (e.g., inhibiting cancer proliferation, inhibiting cancer progression) in a subject in need of treatment comprising administering to the subject any membrane-inserting amphiphilic ligand as described herein, any engineered immune cell as described herein, or any pharmaceutical composition described herein. In some embodiments, the present disclosure provides technologies for treating a solid tumor. In some embodiments, the present disclosure provides technologies for treating a cancer, as described herein.

In some embodiments, the present disclosure provides methods for treating a tumor (e.g., a solid tumor, a tumor of a subject in need of treatment) and/or a cancer. In some such embodiments, a method of treating a tumor comprises introducing (e.g., administering) a membrane-inserting amphiphilic ligand as described herein to a subject in need of treatment followed by administering an engineered immune cell that specifically binds to the amphiphilic ligand as described herein, or a pharmaceutical composition described herein. In some embodiments, treatment locally at a primary tumor results in a systemic anti-tumor immune response, which can result in treatment of a secondary tumor (e.g., a tumor distal to the primary tumor).

In some embodiments, membrane-inserting amphiphilic ligands as described herein, engineered immune cells as described herein, and/or pharmaceutical compositions described herein can be used in the development of a targeted immunotherapy for treating a tumor (e.g., a solid tumor, a tumor of a subject in need of treatment) and/or a cancer.

In certain embodiments, the present disclosure provides technologies for preventing cancer in a subject in remission from cancer, comprising administering to the subject in need of treatment any membrane-inserting amphiphilic ligand described herein, any engineered immune cell described herein, or any pharmaceutical composition described herein.

In some embodiments, the cancer is a relapsed cancer. In some embodiments, the cancer is a refractory cancer. In some embodiments, the cancer is an advanced stage cancer. In some embodiments, the cancer is resistant to one or more other therapies (e.g., chemotherapy, radiotherapy, stem cell transplantation, or another immunotherapy).

The effectiveness of any therapy described herein can be assessed by evaluating a parameter (e.g., tumor burden) before and after administration of the therapy (e.g., to the subject in need of treatment). Any assay known in the art can be used to evaluate the therapeutic effectiveness of the therapies described herein.

### Methods of Administration

Technologies of the present disclosure (e.g., compositions, therapies) can be administered to a subject (e.g., a subject in need of treatment) by any suitable means which include, but are not limited to, parenteral route of administration. In some embodiments, compositions as described herein are administered via the same route of administration. In some embodiments, compositions described herein are administered via different routes of administration.

In some embodiments, composition(s) as described herein are administered to the patient parenterally. Non-limiting examples of parenteral administration include intravenous, intramuscular, intraarterial, subcutaneous, intratumoral, intrathecal and intraperitoneal administration. In one embodiments, a composition(s) described herein is administered intratumorally. In one embodiment, a composition(s) described herein is administered intravenously. In one embodiment, a composition(s) described herein is administered intraperitoneally. In one embodiment, a composition(s) described herein is administered intramuscularly. In one embodiment, a composition(s) described herein is administered subcutaneously. In certain embodiments, the administration is intravenous, intrathecal, intraosseous or into the spinal cord. In one embodiment, a composition(s) described herein is administered into the spinal cord or the spinal canal. In one embodiment, a composition(s) described herein is administered intrathecally. In one embodiment, a composition(s) described herein is administered intraosseously. In one embodiment, a composition(s) described herein is administered into the bone marrow.

In some embodiments, a membrane-inserting amphiphilic ligand as described herein is administered intratumorally. Intratumoral (i.t.) administration of a membrane-inserting amphiphilic ligand described herein can result in the membrane-inserting amphiphilic ligand remaining local after i.t. administration (e.g., in the tumor tissue in which it was administered without spreading or without significant spreading to neighboring tissue, such as non-tumor tissue). In some embodiments, a membrane-inserting amphiphilic ligand is administered intratumorally to a primary tumor. Without wishing to be bound by any one theory, it is understood that such local administration (e.g., i.t. administration) of a membrane-inserting amphiphilic ligand to a primary tumor results in redirection of an engineered immune cell to the primary tumor and can also lead to a systemic anti-tumor immune response, thus, resulting in the treatment of secondary tumors (e.g., a tumor at a site distal to the primary tumor).

In some embodiments, a membrane-inserting amphiphilic ligand as described herein is administered subcutaneously. In some such embodiments, the membrane-inserting amphiphilic ligand is introduced into dendritic cells in lymph nodes of the subject by subcutaneous injection. In some embodiments, a membrane-inserting amphiphilic ligand as described herein is administered intratumorally and subcutaneously (e.g., into dendritic cells in lymph nodes of the subject by subcutaneous injection).

In some embodiments, an engineered immune cell as described herein is administered by systemic infusion.

An appropriate dosage of membrane-inserting amphiphilic ligand and/or engineered immune cells as described herein will vary with the particular condition and/or disease (e.g., a tumor, a cancer) being treated, the age, weight, and physical condition of the subject in need of treatment, the severity of the cancer, the route of administration, the duration of the treatment, the responsiveness of the subject being treated, the nature of the concurrent or combination therapy (if any), the specific route of administration and like factors within the knowledge and expertise of a health practitioner. In certain embodiments, a maximal tolerable dose of a membrane-inserting amphiphilic ligand as described herein and/or an engineered immune cell as described herein is to be used, that is, the highest safe dose according to sound medical judgement. In preferred embodiments, a membrane-inserting amphiphilic ligand and/or an engineered immune cell as described herein is to be administered in effective amounts. An effective amount is, for example, a dose of the composition(s) sufficient to provide a medically desirable result. A medically desirable result, for example, for a subject in need of treatment with a tumor, may be an amount that decreases tumor volume or load. Effective amounts may also be assessed by the presence and/or frequency of tumor cells in the blood or other body fluid or tissue. If a tumor impacts the standard functioning of a tissue or organ, the effective amount may be assessed by measuring standard functioning of the tissue or organ.

In certain embodiments, a membrane-inserting amphiphilic ligand as described herein is administered into a tumor of a subject in an amount of about or at least 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 20 nM, 25 nM, 50 nM, 75 nM, 100 nM, 150 nM, 200 nM, 250 nM, 500 nM, 750 nM, or 1 μM, 5 μM, 10 μM, 20 μM, 30 μM, 40 μM, 50 μM, 60 μM, 70 μM, 80 μM, 90 μM, 100 μM, 200 μM, 300 μM, 400 μM, 500 μM, 750 μM or 1 mM (or any value or range in between). In certain embodiments, for example, a membrane-inserting amphiphilic ligand as described herein is administered into a tumor of a subject in an amount of about or at least 1 nM to 1 mM, 10 nM to 1 mM, 100 nM to 1 mM, 250 nM to 1 mM, 500 nM to 1 mM, 750 nM to 1 mM, 1 nM to 750 μM, 1 nM to 500 μM, 1 nM to 250 μM, 1 nM to 100 μM, or 1 nM to 10 μM.

In certain embodiments, an engineered immune cell as described herein are administered in an amount of about or at least 1×10⁴, 5×10⁴, 1×10⁵, 5×10⁵, 1×10⁶, 5×10⁶, 1×10⁷, 5×10⁷, 1×10⁸, 1×10⁹, 1×10¹⁰, 5×10¹⁰, 1×10¹¹, or 5×10¹¹, 1×10¹², or 5×10¹² cells (or any value or range in between).

Various dosing schedules of the compositions described herein are contemplated including single administration or multiple administrations over a period of time. The methods of administration include, without limitation, bolus administration and infusions (e.g., continues or pulse infusions).

The therapeutic regimen for use in the methods described herein may include administration of a composition as described herein once a day, once every two days, once every three days, once every four days, once every five days, once every six days, twice a week, once every week, once every two weeks, once every three weeks, once every month or 4 weeks, once every six weeks, once every two months or eight weeks, or once every three months or twelve weeks. In certain embodiments, the subject receives a single dose of any therapy described herein. In certain embodiments, the subject receives from at least two, at least three, at least four, at least five, at least six, at least eight, or at least ten doses of any therapy described herein. In certain embodiments, a therapy described herein is administered daily, every other day, or two times a week. In certain embodiments, a therapy described herein is administered for a period of time, such as one week, two weeks, three weeks, four weeks, six weeks, two months, three months, four months, five months, six months, or one year.

In some embodiments, the initial treatment period (where the therapy is administered, e.g., a single time, twice a week, once a week, twice in two weeks, or once a month) is followed by a withdrawal period in which the antibody is not administered (for, e.g., a week, two weeks, three weeks, 1 month or four weeks, six weeks, two months or 8 weeks, three months, four months, five months, six months, or 1 year), and then followed by a second treatment period (where the therapy is administered, e.g., a single time, twice a week, once a week, twice in two weeks, or once a month). Such initial treatment and such second treatment periods can last, for example, two weeks, three weeks, four weeks, six weeks, two months, or three months (where the initial treatment period can be the same or different from the second treatment period).

### Patient Populations

In some embodiments, a subject in need of treatment in accordance with the technologies disclosed herein include, but are not limited to, humans and non-human vertebrates. In some embodiments, a subject in need of treatment in accordance with the technologies disclosed herein comprise, for example, a mammal. In some such embodiments, a mammal includes, for example and without limitation, a household pet (e.g., a dog, a cat, a rabbit, a ferret, a hamster, etc.), a livestock or farm animal (e.g., a cow, a pig, a sheep, a goat, a pig, a chicken or another poultry), a horse (e.g., a thoroughbred horse), a monkey, a laboratory animal (e.g., a mouse, a rat, a rabbit, etc.), and the like. Subjects also include fish and other aquatic species. In a preferred embodiment, the subject being treated in accordance with the methods described herein is a human. In some embodiments, technologies of the present disclosure can be practiced in any subject in need of treatment that is likely to benefit from targeted immunotherapy for the treatment of a tumor, including, for example, a solid tumor.

In some aspects, the technologies disclosed herein can be practiced in any subject that has (e.g., has been diagnosed with) a cancer that may (or is likely to) benefit from any immunotherapy described herein. A subject having a tumor (e.g., a solid tumor) is a subject that has detectable tumor cells. The disclosure contemplates, among other things, any membrane-inserting amphiphilic ligand described herein and/or any engineered immune cell described here to a subject in need of treatment (e.g., a subject with a tumor).

In some aspects, the therapeutic methods and uses of the present disclosure can be practiced in a subject that has cancer. A subject that has cancer is a subject that has detectable cancer cells. Certain exemplary cancers that may, in some embodiments, be treated in accordance with the present disclosure include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromocytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some embodiments, a cancer may involve one or more tumors. In some embodiments, a tumor comprises a solid tumor. In some embodiments, solid tumors include but are not limited to tumors of the bladder, breast, central nervous system, cervix, colon, esophagus, endometrium, head and neck, kidney, liver, lung, ovary, pancreas, skin, stomach, uterus, or upper respiratory tract.

Tests for diagnosing the cancers to be treated by the methods described herein are known in the art and will be familiar to the ordinary medical practitioner. These laboratory tests include, without limitation, microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner generally takes a full history and conducts a complete physical examination in addition to running the laboratory tests listed above.

In some aspects, the subject being treated has previously undergone one or more other cancer therapies (e.g., chemotherapy, radiotherapy, or stem cell transplantation). In certain embodiments, the subject being treated has previously undergone one or more other cancer therapies (e.g., chemotherapy, radiotherapy, or stem cell transplantation), and the subject's cancer has relapsed. In certain embodiments, the subject being treated has previously undergone one or more other cancer therapies (e.g., chemotherapy, radiotherapy, or stem cell transplantation), and the subject has developed resistance to the one or more other cancer therapies. In certain embodiments, the subject being treated is in remission (e.g., in partial remission or in complete remission of cancer). In certain embodiments, the subject being treated is refractory to one or more other cancer therapies (e.g., chemotherapy, radiotherapy, or stem cell transplantation).

In other embodiments, contemplated herein is treating a subject that is at risk of developing cancer that may (or is likely to) benefit from any immunotherapy described herein in accordance with the therapeutic methods and uses of the disclosure. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission. The disclosure contemplates administration of membrane-inserting amphiphilic ligands described herein and engineered immune cells as described herein, or compositions comprising the same, to subjects at risk of developing a cancer.

In on embodiment, the subject in need of treatment is an adult. In one embodiment, the subject is a human subject over 18 years of age. In one embodiment, the subject is a human subject over 21 years of age. In one embodiment, the subject is a human subject over 45 years of age. In one embodiment, the subject is a human subject over 65 years of age. In one embodiment, the subject is a human subject under 18 years of age. In one embodiment, the subject is a human subject under 45 years of age (or between 18 and 45 years of age, or between 21 and 45 years of age). In one embodiment, the subject is a human subject under 65 years of age (or between 18 and 65 years of age, between 21 and 65 years of age, or between 45 and 65 years of age).

### Combination Therapies

In some aspects, therapeutic methods and uses described herein further include methods of treating a tumor (e.g., a tumor of a subject in need of treatment) with additional agents that enhance therapeutic responses, such as, for example, enhance an anti-tumor response in a subject in need of treatment and/or that are cytotoxic to the tumor (e.g., chemotherapeutic agents).

In some aspects, a method of treatment described herein is administered to a subject in combination with one or more anti-cancer therapy, e.g., a chemotherapy, a radiation therapy, stem cell transplantation, a small molecule with an anti-cancer activity, another anti-cancer immunotherapy (e.g., another anti-cancer antibody or fragment thereof, or another T cell therapy), or any other anti-cancer therapy known in the art.

In some aspects, one or more of the membrane-inserting amphiphilic ligands, or compositions comprising the same, as described herein, are administered to a subject in need of treatment in combination with one or more anti-cancer therapy, e.g., a chemotherapy, a radiation therapy, stem cell transplantation, a small molecule with an anti-cancer activity, another anti-cancer immunotherapy (e.g., an anti-cancer antibody or fragment thereof, or another T cell therapy), or any other anti-cancer therapy known in the art.

In some aspects, one or more of the engineered immune cells, or compositions comprising the same, as described herein are administered to a subject in need of treatment in combination with stem cell transplantation or another anti-cancer immunotherapy (e.g., an anti-cancer antibody or fragment thereof, or another cell therapy). For administration with engineered immune cells, any combination of therapies that would not negatively affect the viability of the engineered immune cells are contemplated herein.

In some aspects, one or more of the membrane-inserting amphiphilic ligands and engineered immune cells, or compositions comprising the same, as described herein are administered to a subject in need of treatment in combination with stem cell transplantation or another anti-cancer immunotherapy (e.g., an anti-cancer antibody or fragment thereof, or another cell therapy). For administration with engineered immune cells, any combination of therapies that would not negatively affect the viability of the engineered immune cells are contemplated herein.

Suitable therapeutic agents for use in combination therapy include small molecule chemotherapeutic agents, including protein tyrosine kinase inhibitors, as well as biological anti-cancer agents, such as anti-cancer antibodies, including but not limited to those discussed further below.

In some aspects, combination therapy includes administering to the subject an immune checkpoint inhibitor to enhance anti-tumor immunity, such as a PD-1 inhibitor, a PD-L1 inhibitor, a PD-L2 inhibitor, or a CTLA-4 inhibitor. Other modulators of immune checkpoints may target TIM-3, OX-40, OX-40L or ICOS. In one embodiment, an agent that modulates an immune checkpoint is an antibody (e.g., an antagonistic antibody to PD-1, PD-L1, PD-L2, CTLA-4, TIM-3, or OX-40). In another embodiment, an agent that modulates an immune checkpoint is a protein or small molecule modulator. In another embodiment, the agent (such as an mRNA) encodes an antibody modulator of an immune checkpoint. In one embodiment, any therapy described herein is administered in combination with a TIM-3 inhibitor. In one embodiment, any therapy described herein is administered in combination with a PD-1 inhibitor. In one embodiment, any therapy described herein is administered in combination with a PD-L1 inhibitor. In one embodiment, any therapy described herein is administered in combination with a CTLA-4 inhibitor. Non-limiting examples of immune checkpoint inhibitors that can be used in combination therapy include pembrolizumab, alemtuzumab, nivolumab, pidilizumab, ofatumumab, rituximab, MEDIO680, PDR001, AMP-224, PF-06801591, BGB-A317, REGN2810, SHR-1210, TSR-042, affimer, avelumab (MSB0010718C), atezolizumab (MPDL3280A), durvalumab (MEDI4736), BMS936559, ipilimumab, tremelimumab, AGEN1884, MEDI6469 and MOXR0916.

In a specific embodiment, a therapy described herein is administered to a subject in combination with chemotherapy. Examples of types of chemotherapeutic agents that can be used in the combination therapy described herein include, without limitation, an alkylating agent, a nitrosourea agent, an antimetabolite, a platinum complex derivative, a topoisomerase inhibitor, an aromatase inhibitor, an alkaloid derived from a plant, a hormone antagonist, an antitumor antibiotic, and a P-glycoprotein inhibitor. Specific examples of chemotherapeutic drugs that can be used in the combination therapy described herein include, without limitation, taxol, paclitaxel, nab-paclitaxel, 5-fluoro uracil (5-FU), gemcitabine, doxorubicin, daunorubicin, colchicin, mitoxantrone, tamoxifen, cyclophosphamide, mechlorethamine, melphalan, chlorambucil, busulfan, uramustine, mustargen, ifosamide, bendamustine, carmustine, lomustine, semustine, fotemustine, streptozocin, thiotepa, mitomycin, diaziquone, tetrazine, altretamine, dacarbazine, mitozolomide, temozolomide, procarbazine, hexamethylmelamine, altretamine, hexalen, trofosfamide, estramustine, treosulfan, mannosulfan, triaziquone, carboquone, nimustine, ranimustine, azathioprine, sulfanilamide, fluoropyrimidine, thiopurine, thioguanine, mercaptopurine, cladribine, capecitabine, pemetrexed, fludarabine, methotrexate, hydroxyurea, nelarabine or clofarabine, cytarabine, decitabine, pralatrexate, floxuridine, thioquanine, azacitidine, cladribine, pentostatin, mercaptopurine, imatinib, dactinomycin, cerubidine, bleomycin, actinomycin, luteomycin, epirubicin, idarubicin, plicamycin, vincristin, vinblastine, vinorelbine, vindesine, vinflunine, paclitaxel, docetaxel, etoposide, teniposide, periwinkle, vinca, taxane, irinotecan, topotecan, camptothecin, teniposide, pirarubicin, novobiocin, merbarone, aclarubicin, amsacrine, antiandrogen, anti-estrogen, bicalutamide, medroxyprogesterone, fluoxymesterone, diethylstilbestrol, estrace, octreotide, megestrol, raloxifene, toremifene, fulvestrant, prednisone, flutamide, leuprolide, goserelin, aminoglutethimide, testolactone, anastrozole, letrozole, exemestane, vorozole, formestane, fadrozole, androstene, resveratrol, myosmine, catechin, apigenin eriodictyol isoliquiritigenin, mangostin, amiodarone, azithromycin, captopril, clarithromycin, cyclosporine, piperine, quercetine, quinidine, quinine, reserpine, ritonavir, tariquidar, verapamil, cisplatin, carboplatin, oxaliplatin, transplatin, nedaplatin, satraplatin, triplatin and carboplatin.

In a specific embodiment, a therapy described herein is administered to a subject in combination with radiation therapy.

In a specific embodiment, any therapy described herein is administered to a subject in combination with stem cell transplantation.

In certain embodiments, any therapy described herein can be administered before, during (i.e., concurrently) or after one or more additional anti-cancer therapy. In one embodiment, the subject being treated in accordance with the methods described herein has not previously received an anti-cancer therapy. In one embodiment, the subject being treated in accordance with the methods described herein has previously received an anti-cancer therapy (e.g., a chemotherapy, a radiation therapy, or a stem cell transplant).

### Kits

In one aspect, provided herein are kits comprising one or more containers comprising (i) a membrane-inserting amphiphilic ligand as described herein, a CAR polypeptide described herein, engineered immune cell (e.g., a T cell) comprising a CAR polypeptide described herein, or a pharmaceutical composition described herein; (ii) optionally, one or more additional anti-cancer agents (e.g., a chemotherapeutic agent), and (iii) optionally, instructions for use in treating a tumor.

In certain embodiments, the disclosure pertains to kits comprising a membrane-inserting amphiphilic ligand as disclosed herein, and instructions for use. In certain embodiments, the disclosure pertains to kits comprising an engineered immune cell (e.g., a T cell) comprising a CAR polypeptide described herein, and instructions for use. In certain embodiments, the disclosure pertains to kits comprising an engineered immune cell as disclosed herein, and instructions for use.

In some embodiments, a kit may comprise, in the same or separate suitable containers a membrane-inserting amphiphilic ligand as disclosed herein and a pharmaceutically acceptable carrier (e.g., a buffer). In some embodiments, a kit may comprise in the same or separate suitable containers, an engineered immune cell as disclosed herein and a pharmaceutically acceptable container (e.g., a buffer). In some embodiments, a kit may comprise, in the same or separate suitable containers, a membrane-inserting amphiphilic ligand as disclosed herein, an engineered immune cell as disclosed herein, and a pharmaceutically acceptable carrier (e.g., a buffer).

A suitable container may include, without limitation, a vial, well, test tube, flask, bottle, syringe, infusion bag, or other container means, into which the membrane-inserting amphiphilic ligand described herein or engineered immune cell as described herein, may be placed (and in some instances, suitably aliquoted). Where an additional component is provided, the kit can contain additional containers into which this component may be placed. The containers may further include injection or blow-molded plastic containers in which the desired vials are retained. Containers and/or kits can include labeling with instructions for use and/or warnings.

### EXAMPLES

### Example 1: Materials and Methods

The following materials and methods were used for the following Examples.

Materials: DSPE-PEG-FITC (PEG MW 1, 2, 3.4, 5 kDa) was purchased from Creative PEGworks (cat #PLS-9926 to 9929). DSPE-FITC and DSPE-PEG10k-FITC were purchased from Nanosoft Polymers (cat #10805) and Nanocs (cat #PG2-DSFC-10k), respectively. Cyclic di-GMP was purchased from Invivogen.

Cell lines: Phoenix-ECO, B16F10, and MSTO-211H cells were purchased from ATCC. 293T cells were purchased from Clontech. MC38 and CT-2A-Luc cells were kindly gifted. CT-2A and MSTO-211H cells were lentivirally transduced to stably express murine EGFRvIII and tdTomato or human mesothelin, respectively, with selection using puromycin and sorting on a BD FACSAria™ III Cell Sorter. B16F10, CT-2A, MC38, 293T, and Phoenix-ECO cells were cultured in complete Dulbecco's modified Eagle's medium (DMEM, Cytiva; supplemented with 10% FBS, 100 U/mL penicillin, and 100 μg/mL streptomycin). MSTO-211H cells were cultured in complete RPMI medium (Cytiva).

Construct design: All murine CARs contained the CD8α signal peptide (MALPVTALLLPLALLLHAARP) followed by a Myc-tag (EQKLISEEDL), for analysis of cell surface expression) and a FITC-specific scFv derived from monoclonal antibody clone 4m5.3, E2, or E2 with a His-H58-Ala mutation (“E2.7”) (see, e.g., Boder, E. T et al. Proceedings of the National Academy of Sciences of the United States of America 97, 10701-10705 (2000); Vaughn, T. J. et al., Nature Medicine 2, 534-539 (1996)); Pedrazzi, G. et al., FEBS Letters 415, 289-293 (1997)). Extracellular domains were fused to the CD8α hinge and transmembrane domains, CD28 or 4-1BB costimulatory domains, and a CD3ζ intracellular domain, and were cloned into a pMIG retroviral vector. For BLI studies, T cells were co-transduced with MI-FLuc-IRES-mCherry (Addgene plasmid #75020; http://n2t.net/addgene:75020; RRID:Addgene_75020). All humanized CARs were similarly cloned as above using humanized domains, with the exception that CARs containing CD28 costimulatory domains were paired with a CD28 transmembrane domain. Humanized CARs were cloned into pLenti6.3 lentiviral vector (ThermoFisher) under the control of a cytomegalovirus (CMV) promoter. For BLI studies, T cells were co-transduced with pCDH-EF1-Luc2-P2A-tdTomato (Addgene plasmid #72486; http://n2t.net/addgene:72486; RRID:Addgene_72486). Expression of all CARs was determined by flow cytometry staining for the Myc-tag using an anti-Myc tag antibody (9B11, PE conjugate, Cell Signaling) with analysis on a BD LSR-II flow cytometer.

Retrovirus production and engineering of murine CAR T cells: To produce ecotropic retrovirus for murine T cell transduction, Phoenix-ECO cells were transfected with a 1:3 ratio of pCL-Eco and transfer plasmid using a CalPhos Mammalian Transfection Kit (Takara Bio). Primary murine T cells were isolated from spleens of WT or CD45.1+ C57BL/6 mice using the EasySep Mouse CD8+ T Cell Isolation Kit or EasySep Mouse T cell Isolation Kit (StemCell Technologies) and activated for 48 hours on 6-well non-TC-treated plates precoated with 0.5 μg/mL anti-CD3 (BioXCell, Clone 2C11) and 5 μg/mL anti-CD28 (BioXCell, Clone 37.51) at 10⁶ cells/mL with 3-5 mL/well. T cells were cultured in complete RPMI with 1 mM sodium pyruvate, 0.05 mM β-mercaptoethanol, and 1× minimal essential medium (MEM) non-essential amino acids (ThermoFisher) supplemented with 10 ng/mL murine IL-2 (BioLegend). Following activation, T cells were transduced by spinfection at 3×10⁶ cells/well in complete T cell media for 90 min at 1100×g and 32° C. with 10 ng/mL polybrene (Sigma) on non-TC-treated plates precoated with 15 μg/mL RetroNectin (Takara Bio). CAR expression was assayed via flow cytometry 24 hr post-transduction; T cells were maintained at a cell density of 10⁶ cells/mL, then used for adoptive transfer or in vitro assays at 48 hr post-transduction.

Lentivirus production and engineering of human CAR T cells: For humanized CARs, vesicular stomatitis virus G (VSV-G)-pseudotyped lentivirus was produced via transfection of 293T cells with psPAX2, pMD2.G, and transfer plasmid in a 2:1:2 ratio using Effectene Transfection Reagent (QIAGEN). Primary human T cells were isolated from buffy coats from healthy donors (Massachusetts General Hospital Blood Donor Center) using the EasySep Human CD8+ T Cell Isolation Kit or EasySep Human T Cell Isolation Kit (StemCell Technologies) and activated for 48 hours using Dynabeads Human T-Activator CD3/CD28 (ThermoFisher) at a 3:1 bead-to-cell ratio. Human T cells were cultured in complete RPMI supplemented with 30 U/mL human IL-2 (PeproTech). Following activation, T cells were transduced and stained as described above, and used for adoptive transfer or in vitro studies at 48-72 hr post-transduction.

Amph-FITC labeling and cytotoxicity studies: For in vitro labeling of tumor cells with amph-FITC, tumor cells were washed with 1×PBS and incubated at a cell density of 10⁶ cells/mL for 30 minutes at 37° C. with DSPE-PEGx-FITC, where x=0, 1, 2, 3.4, 5, or 10 kDa (Creative PEGworks) in PBS. For cocultures, target cells were labeled simultaneously with 100 nM amph-FITC and CellTrace Violet (ThermoFisher); 20,000 target cells were seeded per well in a 96-well flat-bottom plate with CAR T cells at the indicated effector-to-target ratios. Following a 16 hr incubation, cells were stained with SYTOX red (ThermoFisher) for flow cytometry, and IFN-γ in the supernatant was quantified using a Mouse IFN gamma Uncoated ELISA Kit (Invitrogen).

Biodistribution analysis: To assess biodistribution of amph-FITC following intratumoral injection, C57BL/6 mice (6-8 weeks, Jackson Laboratory, n=5) were inoculated subcutaneously with 10⁶ B16F10 tumor cells. Once tumors were ˜25 mm² in area, 10 nmol DSPE-PEG2k-FITC (Creative PEGworks) was injected intratumorally. After 24 hr, tissues (tumor, liver, kidney, spleen, bone marrow, and both axillary and inguinal LNs) were homogenized in ethanol (pH 8.0-8.5) and supernatant fluorescence was quantified on a FlexStation 3 plate reader (Molecular Devices, excitation 495 nm, emission 519 nm).

For cellular localization studies, MC38 tumor-bearing mice were injected intratumorally with 10 nmol DSPE-PEG2k-FITC, and 24 hr later tumors were enzymatically digested at 37° C. for 30 min with 1 mg/mL collagenase D and 0.2 mg/mL DNase I in complete RPMI media before mechanical dissociation through a 70 μm cell strainer. Samples were stained using the following antibodies: PE-Cy7 anti-CD45 (clone 30-F11, BioLegend), BV605 anti-CD11b (clone M1/70, BioLegend), PE anti-CD11c (clone N418, BioLegend), BV711 anti-mouse CD3 (clone 17A2, BioLegend), BV421 anti-CD8α (clone 53-6.7, BioLegend), and Alexa Fluor 647 anti-FITC (Jackson ImmunoResearch) and were analyzed on a BD LSRFortessa flow cytometer.

In vivo therapy studies: All animal work was conducted under a Massachusetts Institute of Technology (MIT) Division of Comparative Medicine institute animal care and use committee-approved protocol in accordance with federal, state and local guidelines.

C57BL/6 WT, CD45.1⁺, Batf3 KO, and Rag1 KO mice (Jackson Laboratory, 6 to 8 weeks old) were inoculated with 10⁶ B16F10, 10⁶ MC38, 10⁶ TC-1, or 3×10⁶ CT-2A tumor cells subcutaneously at the flank. Once tumors grew to ˜25 mm² in area, mice underwent a non-myeloablative 5 Gy dose of TBI administered by a [¹³⁷Cs] gamma radiation source, 24 hr prior to adoptive transfer of 10⁷ CD45.1+ CAR T cells IV via the tail vein. One day later, mice were vaccinated subcutaneously at the tail base with 10 nmol DSPE-PEG2k-FITC (Creative PEGworks) and 25 μg cyclic di-GMP (Invivogen), and vaccinated for 3 total doses, once weekly. Intratumoral injection of 10 nmol amph-FITC was administered beginning the day after the first vaccination, and continuing every 3 days.

For xenograft studies, 6- to 12-week-old NOD.Cg-Prkdc^{scid}IL2rg^{tm1Wj1}/SzJ (NSG) mice (Jackson Laboratory) were inoculated with 3×10⁶ MSTO-211H cells subcutaneously at the flank. Once tumors grew to ˜25 mm², mice were adoptively transferred with 10⁷ CAR T cells IV via the tail vein. Intratumoral injection of 10 nmol amph-FITC was administered beginning the day after adoptive transfer, and continuing every 3 days.

For all studies, tumor progression was monitored by caliper measurements. Survival was evaluated over time until tumors exceeded 200 mm² in area, were severely ulcerated >1 week, or experienced >20% weight loss. To monitor trafficking of Fluc-expressing CAR T cells, mice were injected subcutaneously at the scruff with 150 mg/kg D-luciferin K⁺ Salt (Perkin-Elmer) and imaged on a Xenogen IVIS Spectrum. Prior to imaging, C57BL/6 mice were depilated on their back to improve signal sensitivity.

Flow cytometry: For quantification of murine CAR T and endogenous T cells in the peripheral blood, tumor, and spleen, tissues were homogenized through a 70 μm cell strainer (with the exception of MC38 tumors, which required enzymatic digestion as described above). Blood and spleen samples were incubated with ACK lysing buffer (Gibco) to isolate splenocytes and PBMCs, respectively. Samples were then stained with PE anti-CD45.1 (clone A20, BioLegend), BUV805 anti-CD8α (clone 53-6.7, BioLegend), Alexa Fluor 647 anti-CD4 (clone GK1.5, BioLegend), APC-Cy7 anti-CD45.2 (clone 104, BioLegend), and LIVE/DEAD Fixable Aqua (ThermoFisher) and analyzed on a BD LSRFortessa flow cytometer.

For antigen uptake studies, tumors and LNs were stained with Zombie Aqua Fixable Viability Kit (BioLegend), BUV395 anti-CD45 (clone 30-F11, BD Biosciences), BV421 anti-CD103 (clone 2E7, BioLegend), BV605 anti-Ly6C (clone HK1.4, BioLegend), BV711 anti-F4/80 (clone BMB, BioLegend), BV785 anti-CD11b (clone M1/70, BioLegend), Alexa Fluor 700 anti-CD86 (clone GL1, BioLegend), PE-Cy7 anti-I-A/I-E (clone M5/114.15.2, BioLegend), APC anti-CD24 (clone 30-F1, BioLegend), APC-Cy7 anti-CD11c (clone N418, BioLegend), PE-Cy5 anti-CCR7 (clone 4B12, BioLegend), PerCP-Cy5.5 anti-CD169 (clone 3D6.112, BioLegend), and BUV737 anti-CD8α (clone 53-6.7, BD Biosciences).

For quantification of human CAR T cells in the peripheral blood, spleen, and tumor, tissues were processed as described above, then stained with LIVE/DEAD Aqua, BUV496 anti-CD4 (clone SK3/Leu3a, BD Biosciences), BUV805 anti-CD8α (clone SK1, BD Biosciences), PE anti-myc tag (clone 9B11, Cell Signaling), and APC-Cy7 anti-CD45 (clone 2D1, BioLegend).

ELISPOT: Spleens were processed as described above. CT-2A and B16F10 target cells were pretreated with 100 U/mL and 500 U/mL murine IFN-γ (Abcam), respectively for 16 hr, then irradiated at 120 Gy with a [¹³⁷Cs] gamma radiation source. Splenocytes (several dilutions starting with 10⁶ cells/well) were cocultured with 25,000 target cells/well for 16 hr and ELISPOT was performed using a Mouse IFN-γ ELISPOT Set (BD Biosciences) according to manufacturer's instructions.

Immunohistochemistry and immunofluorescence: Tissues (tumor, lungs, liver, and kidney) were fixed in 4% paraformaldehyde (PFA) in PBS for 24 hr at 4° C. For immunofluorescence studies, tumors were embedded in 3% low gelling temperature agarose (Sigma-Aldrich) before processing into 100 μm sections on a Leica VT1000S vibratome. Tissue sections were permeabilized with 2% BSA, 0.2% Triton-X 100 in PBS for 1 hr at 37° C. prior to staining with Alexa Fluor 647- or DyLight 405-conjugated anti-FITC (Jackson ImmunoResearch) for 16 hr at 37° C., followed by several PBS washes. Confocal microscopy was performed on an Olympus FV1200 Confocal Laser Scanning Microscope. For immunohistochemistry, PFA-fixed samples were paraffin-embedded and 10 μm sections were obtained. Sections were stained with anti-myc tag (9B11, Cell Signaling) or anti-CD45.1 (A20, BioLegend). For immunohistochemistry studies, tumors were frozen in O.C.T. and processed into 14 m thick sections on a Leica CM1950 cryostat. Tissue sections were permeabilized with 2% BSA, 0.2% Triton-X 100 in PBS for 2 hours at room temperature prior to staining with biotinylated anti-Trp1 [TA99] (kindly gifted), BV421-conjugated streptavidin (Biolegend), Alexa Fluor 647-conjugated anti-FITC (clone SPM395, Novus Biologicals), and CellMask™ Orange Plasma Membrane Stain (Invitrogen). Imaging was performed on a Leica SP8 Spectral Confocal Microscope.

Statistical analysis: Statistical analyses were performed using GraphPad Prism 9. All data was presented as mean±SD. To assess statistical significance, the following tests were used: between two groups, unpaired two-tailed Student's t test; for multiple comparisons, two-way ANOVA with Tukey's multiple comparisons; for repeated measures over a time course, two-way ANOVA with repeated measures; for Kaplan-Meier survival curves, log-rank (Mantel-Cox) test. All P values are provided in the figures or in their legends.

### Example 2: Amph-FITC Tags Tumor Cells for Destruction by CAR T Cells

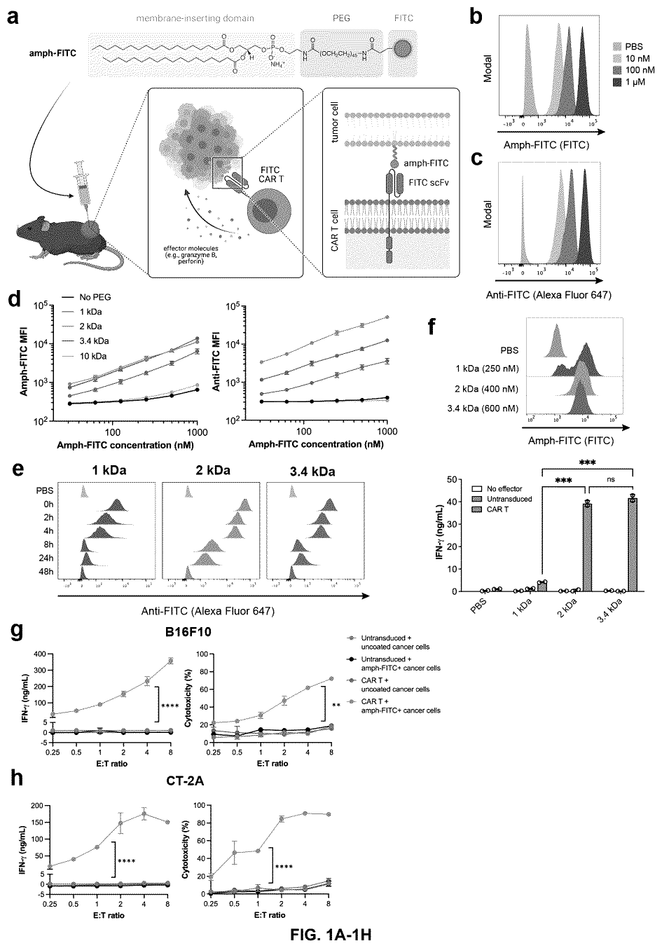

The present example demonstrates Amph-FITC can label diverse cancer cells for effective CAR T cell activation and target cell killing. Exemplary CAR T cell ligand, fluorescein isothiocyanate (FITC), was utilized. FITC is a safe, non-immunogenic compound that can redirect CAR T cells against tumors. It was hypothesized, without wishing to be bound by any one theory, that intratumoral administration of 1,2-distearoyl-sn-glycero-3-phosphoethanolamine-PEG-FITC (DSPE-PEG-FITC, or amph-FITC) would enable recognition and killing of tumor cells by FITC-specific CAR T cells (FIG. 1A).

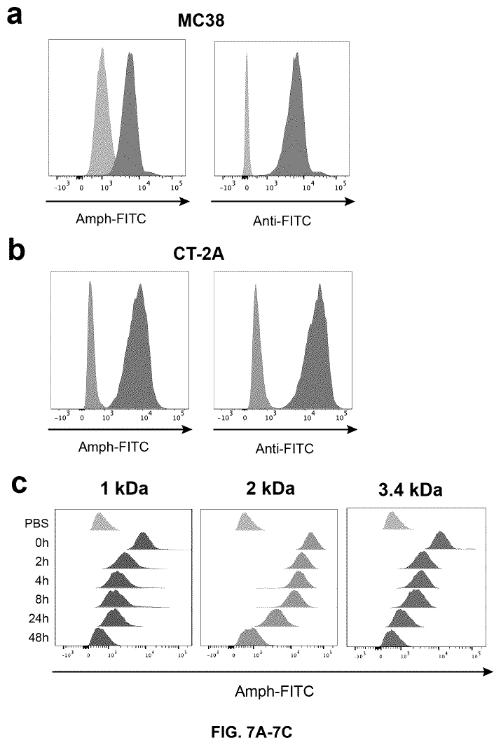

Labeling of cancer cells in vitro was evaluated by incubating amph-FITC with B16F10 murine melanoma cells for 30 min at 37° C. Flow cytometry analysis of the melanoma cells revealed dose-dependent association of amph-FITC with the cells (FIG. 1B), and FITC was exposed on the cell surfaces as revealed by subsequent staining of amph-FITC-coated cells with an anti-FITC antibody (FIG. 1C). Amph-FITC could also associate with and be exposed on the surfaces of MC38 murine colon carcinoma cells and CT-2A glioblastoma cells (FIG. 7A-7B). The physical location of CAR epitopes on target antigens (e.g., closer or farther from the cell membrane) can impact chimeric receptor signaling and CAR T activation (see, e.g., Hudecek, M. et al. Clinical Cancer Research 1, 3153-3165 (2013); Lindner, S. E. et al. https://www.science.org (2020)). In addition, the length of the polymer chain on PEG-DSPE amphiphiles can impact membrane insertion (see, e.g., Liu, H. et al. Nature 507, 519-522 (2014)). To determine how the PEG linker length of amph-FITC affected membrane insertion and stability of labeling, B16F10 cells were incubated with amph-FITC molecules comprised of a range of PEG molecular weights (MW) and the resultant total cell-associated and cell surface-exposed FITC was measured at a range of concentrations (FIG. 1D), then the cells were washed into fresh medium and loss of FITC signal was tracked over 48 hours (FIG. 1E and FIG. 7C). DSPE-PEG2k-FITC achieved the highest insertion at low concentrations and exhibited the greatest persistence, with substantial labeling still detectable after 24 hours in fresh medium.

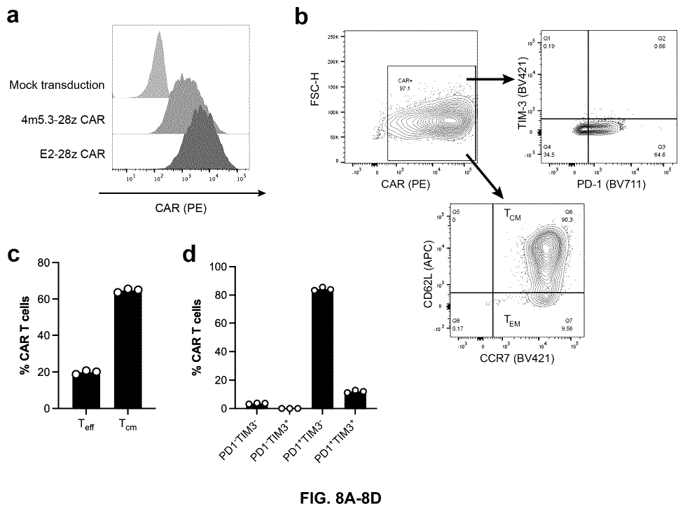

Next, CAR T cell recognition of B16 cells decorated with amph-FITC molecules was tested. FITC-specific CAR T cells were generated by transducing primary murine CD8+ T cells with a CAR comprised of the FITC-specific scFvs 4m5.3 (see, e.g., Boder, E. T. et al. Proceedings of the National Academy of Sciences of the United States of America 97, 10701-10705 (2000)) or E2 (see, e.g., Vaughn, T. J. et al. Nature Medicine 2, 534-539 (1996)) fused to a CD8 hinge, CD8 transmembrane domain, CD28 costimulatory domain, and CD3C signaling domain (abbreviated hereafter as the 4m5.3-28z and E2-28z CARs, FIG. 8A-8D). To assess how PEG linker length influenced the ability of FITC-specific CAR T cells to recognize amph-FITC-decorated target cells, MC38 colon cancer cells were incubated with amph-FITC of varying MW at concentrations titrated to give the same mean number of FITC molecules per cell (FIG. 1F, top), and 4m5.3-28z CAR T cells were added to the labeled tumor cells at a range of effector:target (E:T) ratios. DSPE-PEG2k-FITC and DSPE-PEG3.4k-FITC elicited robust CAR T cell activation as read out by IFN-γ secretion, while the lower MW DSPE-PEG₁ₖ-FITC was much less effective (FIG. 1F, bottom). Based on its relatively high cell membrane insertion and CAR T stimulation properties, DSPE-PEG2K-FITC was utilized for further studies. DSPE-PEG2k-FITC tagging triggered E2-28z CAR T cell activation accompanied by cytokine secretion and efficient killing of B16F10 cells and CT-2A murine glioma cells by FITC CAR T cells (FIG. 1G-1H). Thus, with this exemplary DSPE-PEG2K-FITC molecule, diverse cancer cells can be labeled for effective CAR T cell activation and target cell killing.

### Example 3: Intratumoral Administration of Amph-FITC Labels Tumor Cells

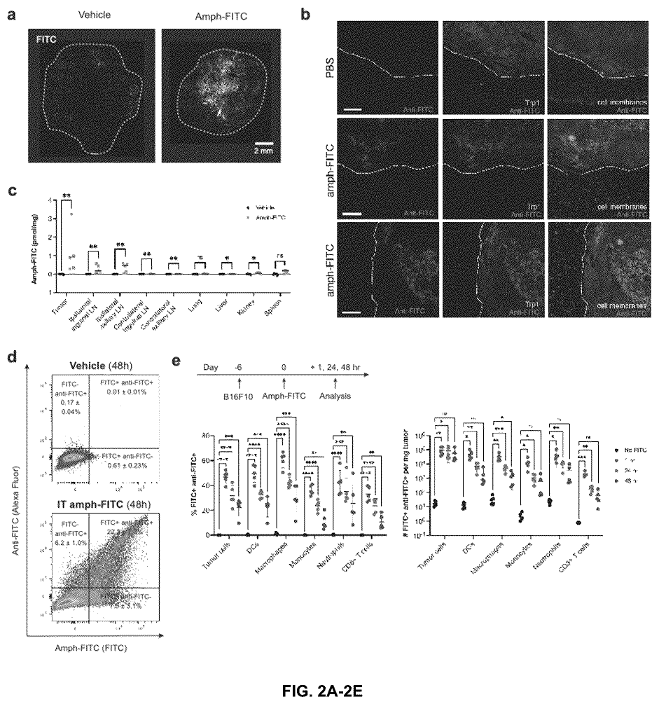

The present example demonstrates intratumoral (i.t.) administration of exemplary membrane-inserting amphiphilic ligand, amph-FITC, labels tumor cells. In vivo tumor labeling following intratumoral injection of amph-FITC was assessed using an amph-FITC dose of 10 nmol. Following a single injection into established B16F10 tumors, use of histology demonstrated that amph-FITC labeled large regions of the tumor (FIG. 2A). To assess potential leakage of amph-FITC outside the tumor into surrounding healthy tissue (e.g., non-tumor), tumor sections and adjacent non-tumor tissue was evaluated by immunohistochemistry. While intratumoral injections did lead to amph-FITC accumulating near the edges of the tumors in some instances, very sparse uptake of amph-FITC on cells in the neighboring tissue was observed (FIG. 2B). Quantification of FITC extracted from the tumor, tumor-draining lymph nodes (TDLNs), and other distal tissues 24 hours post injection showed that amph-FITC remained largely confined to the tumor and draining inguinal and axillary LNs (FIG. 2C). Without wishing to be bound by any one theory, it was hypothesized that amph-FITC uptake into the draining LNs could support CAR T cell proliferation and function by decorating LN-resident APCs.

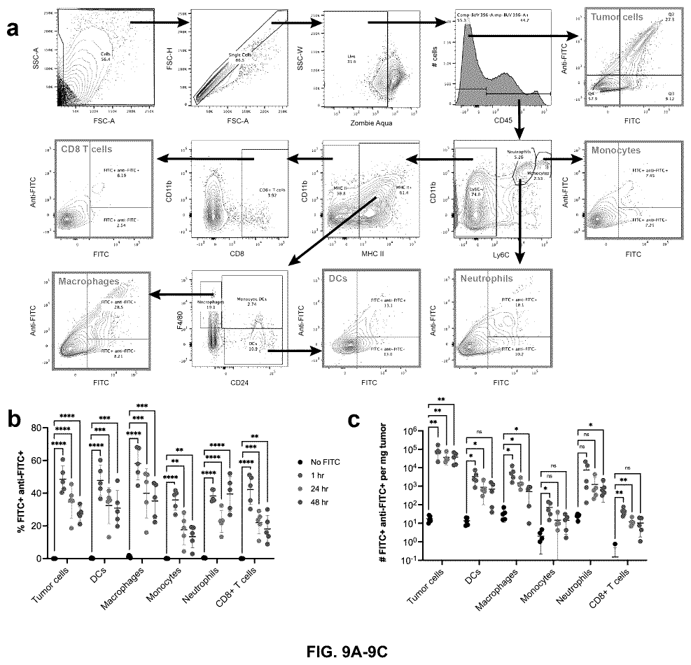

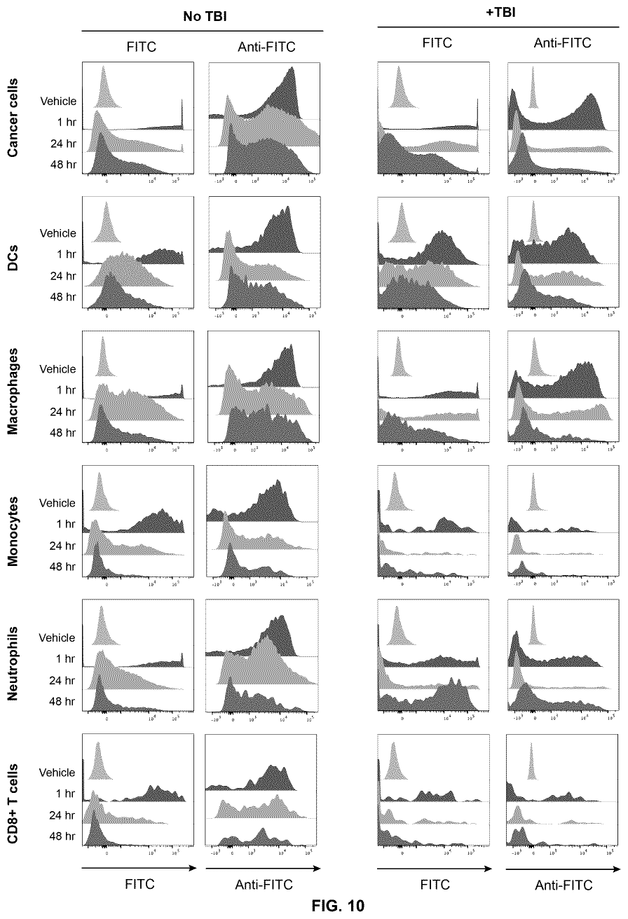

Flow cytometry analysis demonstrated that FITC was taken up by a large proportion of tumor cells 24 hour after injection, and remained accessible at cell surfaces as detected by staining with an anti-FITC antibody (FIG. 2D). Both cancer cells and host cells (including, for example, infiltrating myeloid cells and T cells) were tagged with FITC (FIG. 2E and FIG. 9A), with about 40-60% of each cell type bearing extracellularly exposed FITC after 1 hour, and 20-40% of cells still bearing exposed FITC at 24 hours (FIG. 2E, left and FIG. 10). Numerically by 24 hours the majority of cells surface-decorated with FITC were the cancer cells (FIG. 2E, right). Without wishing to be bound by any one theory, this may be a result of cancer cells relative abundance.

Lymphodepletion (LD) can be used to promote CAR T cell engraftment and could have affected amph-FITC uptake. Thus, analysis of FITC cellular distribution was conducted in animals that received LD by 5 Gy total body irradiation (TBI) 24 hours prior to amph-FITC injection. LD pre-treatment significantly depleted immune cells and favored labeling of cancer cells in an even greater proportion (FIGS. 9B-9C and FIG. 10). Altogether, these data indicate that intratumoral administration effectively restricts amph-FITC availability to the tumor and TDLNs, leading to robust FITC labeling on the surface of cancer cells.

### Example 4: FITC-Specific CAR T Cells Home to and Expand in Tumors

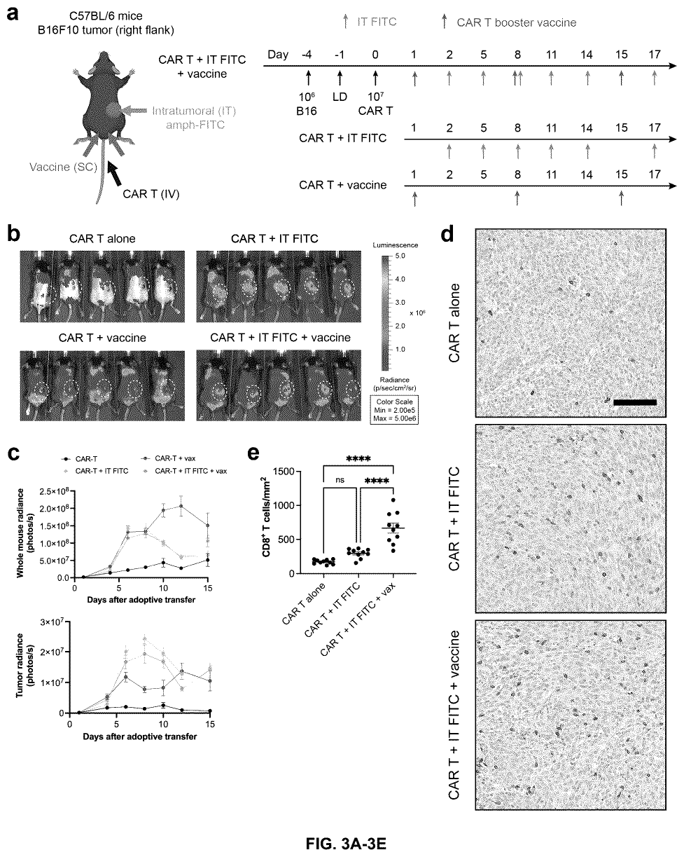

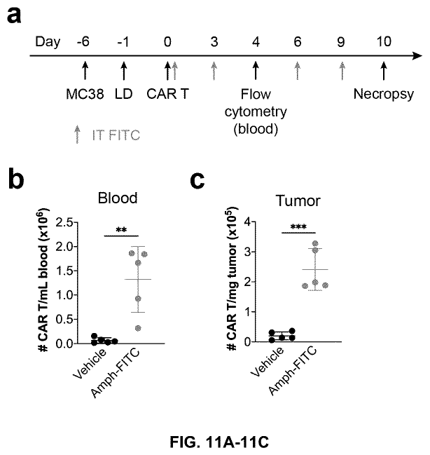

The present example demonstrates that exemplary FITC-specific CAR T cells home to and expand in tumors. The ability of FITC-specific CAR T cells to recognize tumors injected with amph-FITC was characterized and CAR T cell expansion and tumor homing via bioluminescence imaging was evaluated. Mice bearing B16F10 tumors were lymphodepleted by total body irradiation (TBI), followed by adoptive transfer of firefly luciferase-expressing E2-28z CAR T cells. Beginning two days later, amph-FITC was administered intratumorally and every 3 days thereafter (e.g., to re-tag any tumor cells that had not yet been killed by CAR T cells) (FIG. 3A, CAR T+IT FITC). The first dose of i.t. amph-FITC was given after CAR T administration in order to increase the time for the CAR T cells to encounter tumor cells decorated with a high level of the FITC ligand. In a separate group, the effect of CAR T cell transfer (±i.t. amph-FITC) combined with a once-weekly bilateral subcutaneous injection of amph-FITC together with the STING agonist cyclic-di-GMP adjuvant as a vaccine boost was tested (hereafter “amph-FITC vaccination” or “vaccine boost”). This injection targeted amph-FITC to dendritic cells in draining lymph nodes and acted as a vaccine for the CAR T cells, triggering CAR T cell expansion and increased functionality. Bioluminescence imaging of CAR T cells transferred into mice in the absence of amph-FITC treatment revealed a low degree of E2-28z CAR T cell expansion over 14 days without significant infiltration of tumors (FIG. 3B-3C). Administration of i.t. amph-FITC or amph-FITC vaccination individually triggered CAR T cell expansion, but amph-FITC vaccination alone triggered relatively more prominent CAR T cell accumulation near the vaccine injection site near the base of the tail rather than in the tumor. By contrast, intratumoral amph-FITC led to comparable total CAR T cell expansion over the first 7 days, but greater accumulation at the tumor site (FIG. 3B-3C). Addition of the vaccine boost to intratumoral amph-FITC treatment did not have a significant impact on overall T cell expansion or accumulation at the tumor site. This analysis was also conducted by treating CT-2A tumors, and analysis of treated tumors at day 12 revealed substantial T cell infiltration throughout the tumor (FIG. 3D-3E). Without wishing to be bound by any one theory, this was likely by CAR T cells due to the temporal proximity to lymphodepletion. E2-28z CAR T cell infiltration of MC38 colon carcinomas was analyzed, and increases in the abundance of CAR T cells in both the peripheral blood and in the tumor following intratumoral amph-FITC treatment were found (FIG. 11A-11C). Thus, i.t. amph-FITC treatment, both with or without supporting amph-FITC vaccination, lead to significant expansion of the CAR T cell population and infiltration of tumors by E2-28z CAR T cells.

### Example 5: Anti-Tumor Activity of Amph-FITC FITC-Specific CAR T Cell Therapy

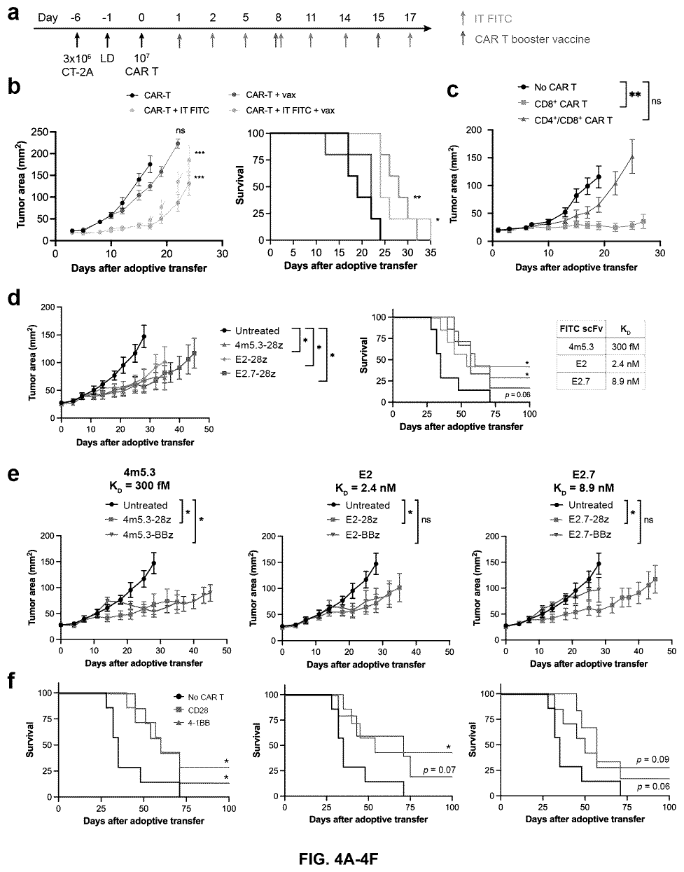

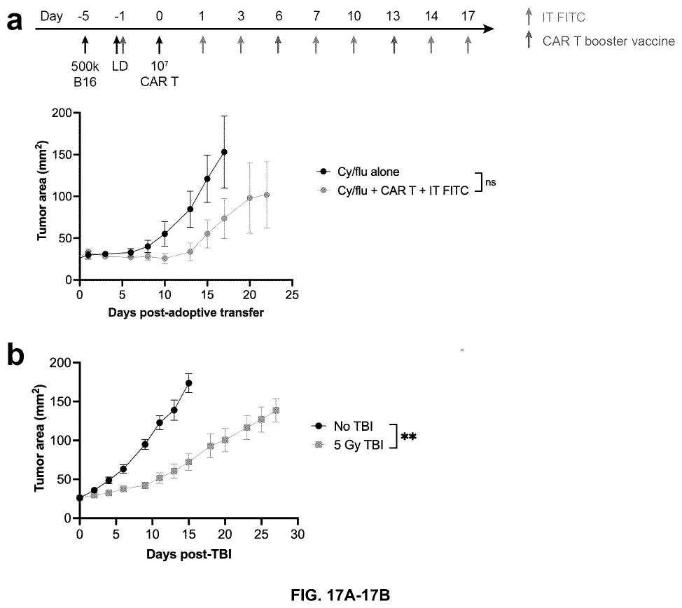

The present example demonstrates anti-tumor activity of exemplary amph-FITC/FITC-specific CAR T cell therapy. The therapeutic impact of FITC-targeting CAR T cell transfer combined with intratumoral amph-FITC and amph-FITC vaccination in the B16F10 and CT-2A tumor models was evaluated following the experimental timelines outline in FIG. 3A and FIG. 4A, respectively. Treatment of the highly aggressive, poorly immunogenic B16F10 model was tested. Treatment was evaluated with or without the inclusion of amph-FITC vaccine boosting. E2-28z CAR T cells redirected by intratumoral amph-FITC halted tumor progression for about 2 weeks and extended survival, while addition of amph-FITC vaccine boosting exhibited a trend toward slightly improved tumor regression and extension of survival, increasing median survival by 3 days (FIG. 4B). To determine whether the method of lymphodepletion impacts response to this therapy, treatment was also tested using the clinical chemotherapy LD regimen of cyclophosphamide and fludarabine prior to CAR T cell transfer, and similar anti-tumor activity was observed (FIG. 17A).

The CT-2A tumor model is a slowly progressing tumor more amenable to immunotherapy treatment. To determine factors governing the therapeutic efficacy of FITC CAR T treatment, the effect of the CD4/CD8 composition in the CAR T cell product was tested. Adoptive transfer of exclusively CD8+ E2-28z CAR T cells combined with intratumoral amph-FITC and vaccination halted progression of CT-2A tumors for 3 weeks (FIG. 4C). Although lymphodepletion alone delayed tumor progression (FIG. 17B), this effect was much weaker than the response elicited by combining adoptive transfer i.t. amph-FITC. Interestingly, in contrast to evidence supporting an important role for CD4+ CAR T cells in humanized mouse models of mesothelioma or lymphoma, transfer of an equivalent number of murine CAR T cells prepared with a ˜1:1 CD8+:CD4+ ratio was substantially less effective (FIG. 4C).

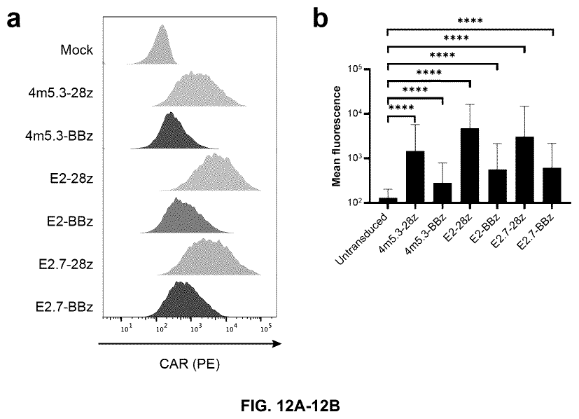

The impact of CAR design parameters (e.g., CAR affinity and costimulatory domain selection) on the efficacy of amph-FITC-redirected CAR T treatment was evaluated. To modulate CAR affinity, the efficacy of CAR T cells bearing the E2 FITC-specific scFv (KD=2.4 nM) with CARs prepared from a very high affinity FITC scFv, 4m5.3 (KD=300 fM), or the lower affinity scFv E2.7 (KD=8.9 nM), which differs from E2 WT by a single amino acid substitution were compared. Each of these three scFv variant CARs was prepared with either a CD28 or 4-1BB costimulatory domain to further interrogate the role of costimulatory signaling. Murine CD8⁺ T cells showed efficient transduction and expression of each of these CARs, though the CD28-based CARs expressed at about 10-fold higher levels than the 4-1BB CARs (FIG. 12A-12B). In tandem with intratumoral amph-FITC+amph-FITC vaccination, all of these FITC-specific CAR T cells displayed similar levels of therapeutic efficacy in treating C2TA tumors and extending animal survival, with the exception of the E2.7-BBz CAR that was slightly less effective (FIG. 4D-4F). However, CARs with CD28 costimulatory domains demonstrated earlier tumor growth control compared to those with 4-1BB costimulatory domains, independent of the affinity of the scFv (FIG. 4E). The most effective E2-28z CAR elicited complete responses in 40% of animals. Based on its therapeutic efficacy and expression levels, this CAR was utilized for subsequent studies,

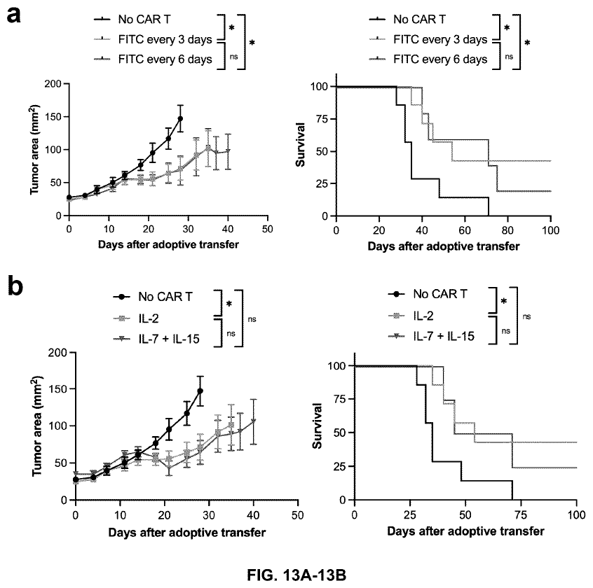

To determine the dosing regimen of amph-FITC and preparation of maximally-effective CAR T cells in this treatment paradigm, the importance of dosing frequency was assessed. Interestingly, reducing injection frequency to once every 6 days did not reduce the therapeutic effect (FIG. 13A). Finally, ex vivo culture of CAR T cells in IL-7 and IL-15, rather than IL-2, typically favors TCM phenotype and increases tumor control (see, e.g., Xu, Y. et al. Blood 123, 3750-3759 (2014); Zhou, J. et al. Protein and Cell 10, 764-769 (2019); Cha, E. et al. Breast Cancer Research and Treatment 122, 359-369 (2010)). However, FITC CAR T cells expanded in IL-7 and IL-15, rather than IL-2, did not demonstrate enhanced tumor control (FIG. 13B). Thus, E2-28z CAR T cells were expanded in IL-2 for further studies.

### Example 6: Amph-FITC and FITC CAR T Therapy is Well-Tolerated with Minimal Toxicity

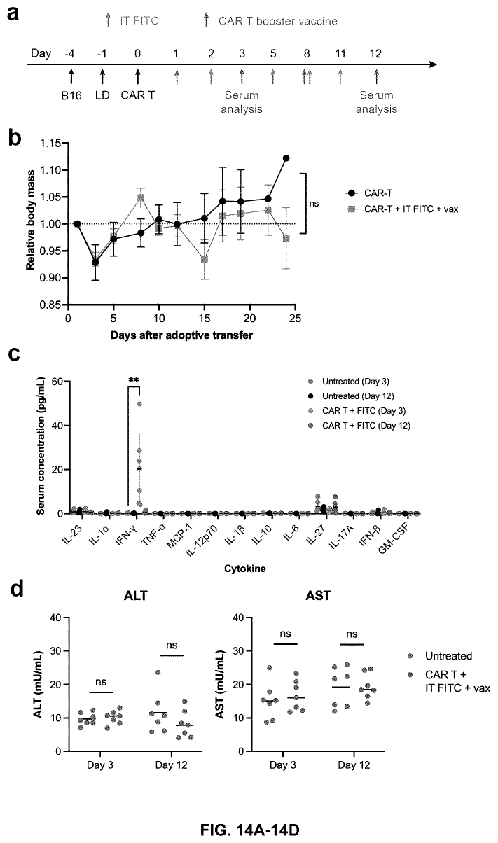

The present example demonstrates exemplary Amph-FITC and FITC CAR T therapy is well-tolerated with minimal toxicity. One concern with the chemical delivery of a CAR T ligand is the potential for dissemination of amph-FITC to trigger CAR T cell attack on healthy tissues. Studies herein demonstrated that local i.t. administration of amph-FITC minimally labeled normal (e.g., non-cancerous) tissues (FIG. 2B) and no obvious inflammation of the skin surrounding treated tumors during treatment was detected, suggesting that the CAR T cell activity remained primarily intratumoral. However, the finding that E2-28z CAR T cells expanded in the peripheral blood following intratumoral amph-FITC treatment (FIG. 11B). Thus, potential systemic toxicities were evaluated. In therapy studies, mice receiving CAR T cells together with intratumoral amph-FITC and amph-FITC vaccination showed no weight loss and gained mass over time indistinguishable from untreated control animals (FIG. 14A-14B). Serum cytokines were measured 24 hours after amph-FITC injection on day 3 or day 12 following E2-28z CAR T cell transfer in the CT-2A tumor model. No elevation of inflammatory cytokines, with the exception of a low level of IFN-γ at day 3, was observed (FIG. 14C). There was no detectable elevation of the liver enzymes, ALT or AST, at corresponding timepoints, indicating a lack of liver toxicity (FIG. 14D). Thus, therapy with intratumoral amph-FITC and FITC-specific CAR T cells appeared to be safe and well-tolerated.

### Example 7: CAR T Cell-Mediated Cytotoxicity Stimulates an Endogenous Anti-Tumor T Cell Response

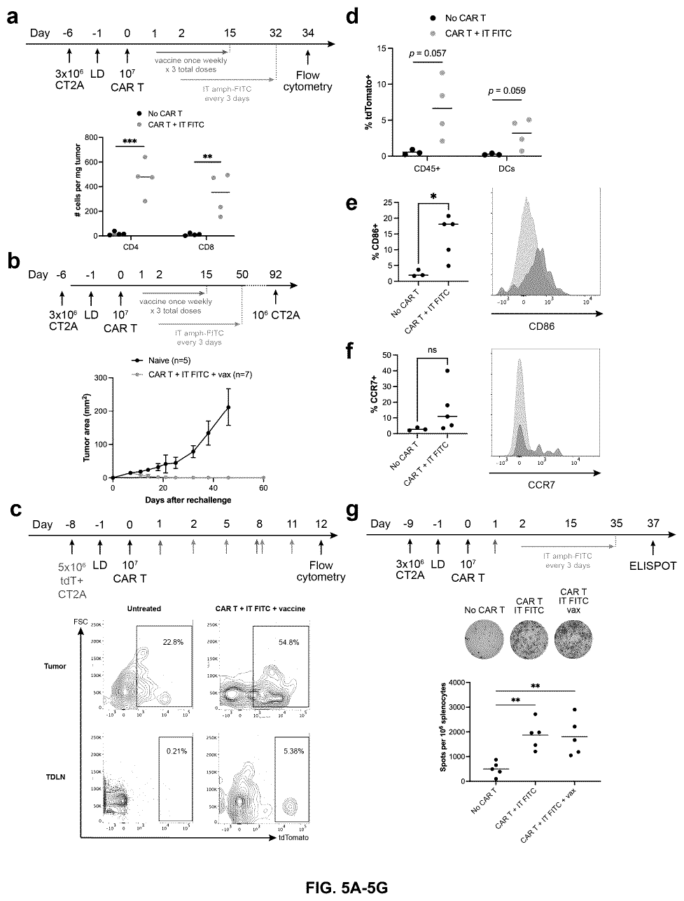

The present example demonstrates CAR T cell-mediated cytotoxicity stimulates an endogenous anti-tumor T cell response. TBI was utilized to promote CAR T cell engraftment at the start of treatment. Infiltration of host CD45.2⁺ CD4⁺ and CD8⁺ T cells into CT-2A tumors treated with intratumoral amph-FITC and CD45.1⁺ CAR T cells was evaluated. While untreated tumors showed very low levels of tumor-infiltrating lymphocytes (TILs), amph-FITC/CAR T treatment elicited robust infiltration by CD45.2⁺ host T cells (FIG. 5A). To assess potential induction of endogenous T cell memory by amph-FITC/CAR T treatment, mice that had rejected CT-2A tumors in response to amph-FITC/E2-28z CAR T therapy were rechallenged with tumor cells on the opposite flank. While control naïve mice inoculated with CT-2A cells showed steady tumor progression, all of the mice cured by amph-FITC/CAR T treatment rejected the rechallenge, despite the absence of FITC in the new tumor (FIG. 5B), suggesting the induction of protective endogenous T cell memory.

An important step for priming of endogenous T cell responses is acquisition of tumor antigen by dendritic cells (DCs). To determine if amph-FITC/CAR T therapy promoted DC uptake of tumor cell debris, tdTomato+ CT-2A tumors were treated and found that immune cells, most notably dendritic cells in both the tumor and tumor-draining lymph node, became tdTomato+, suggesting uptake of tumor antigens (FIG. 5C-5D). Additionally, more DCs in the TDLN expressed activation markers CD86 and CCR7 (FIG. 5E-5F). Redirection of CAR T cells against tumors through amph-FITC decoration promoted antigen delivery to and activation of professional antigen presenting cells that govern endogenous T cell priming. Finally, the development of tumor-specific endogenous T cells following amph-FITC/E2-28z CAR T cell treatment by restimulating splenocytes from amph-FITC/CAR T-treated mice ex vivo with irradiated CT-2A tumor cells was evaluated. Restimulation of splenocytes from treated animals with CT-2A cells confirmed that amph-FITC/CAR T treatment amplified tumor-specific endogenous T cell responses relative to untreated tumor-bearing mice, though addition of amph-FITC vaccine boosting did not appear to provide additional benefit over intratumoral amph-FITC administration alone (FIG. 5G).

### Example 8: Amph-FITC-Mediated CAR T Cell Stimulation Triggers a Systemic Anti-Tumor Immune Response

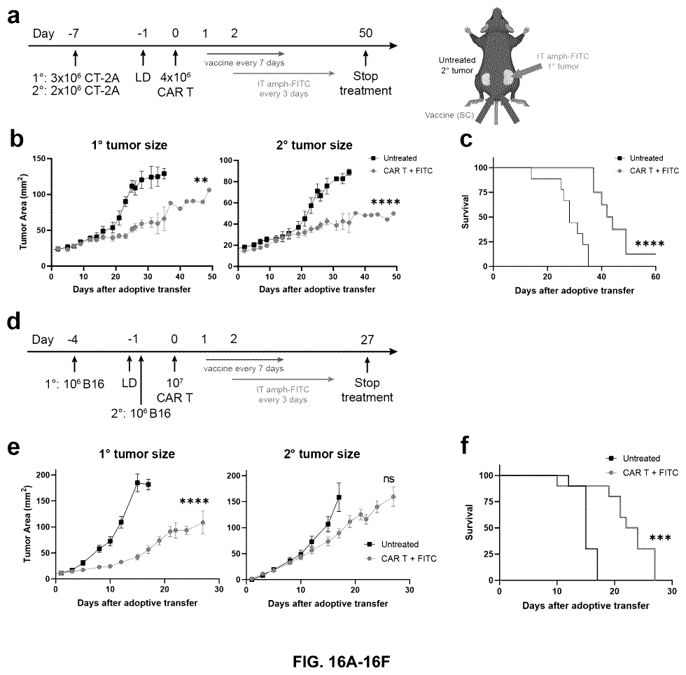

The present example demonstrates that the endogenous T cell response initiated by local redirection of CAR T cells with amph-FITC injection leads to immune attack of distal untreated tumors. In order to assess whether this localized therapy could trigger systemic anti-tumor immune response, CT-2A tumors were implanted bilaterally in the flanks of mice, and only the right flank tumor was treated by administration of i.t. amph-FITC (in combination with amph-FITC vaccination, FIG. 16A). Strikingly, both the injected (1°) and non-injected (2°) tumors showed substantially slowed progression, and overall survival of the treated animals was substantially improved (FIG. 16B-16C). The ability of local amph-FITC delivery to elicit systemic immunity against B16F10 melanomas was tested; for this more aggressive model the un-injected secondary tumor was inoculated several days after the to-be-treated primary tumor, to allow the endogenous T cell response time to develop prior to rampant distal tumor outgrowth (FIG. 16D). Injection of only the primary tumor led to slowed growth of the treated tumor and a trend toward slowed progression of the untreated lesions, in addition to substantial increases in overall survival over untreated animals (FIG. 16E-16F). Thus, the endogenous T cell response initiated by local redirection of CAR T cells with amph-FITC injection leads to immune attack of distal untreated tumors.

### Example 9: Amph-FITC Tagging Enables Human CAR T Cells to Target Xenograft Tumors

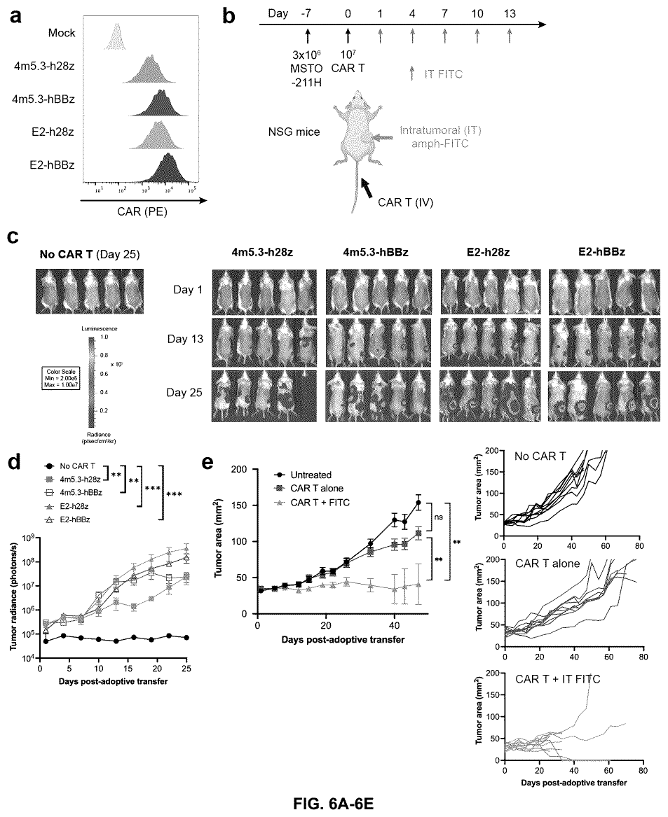

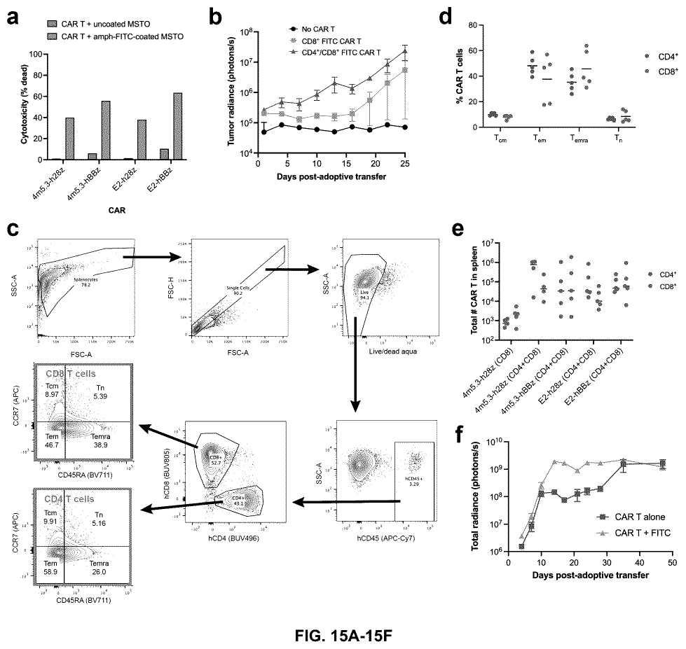

The present example demonstrates Amph-FITC tagging enables human CAR T cells to target xenograft tumors. Studies were conducted in immunocompetent mouse models using murine CAR T cells to enable analysis of antigen spreading and endogenous T cell immunity. The ability of amph-FITC tagging to effectively redirect human CAR T cells against human cancer cells was evaluated using an engineered FITC-specific CARs that were highly expressed in human T cells (FIG. 6A). In vitro, human CAR T cells prepared with either high affinity 4m5.3 or lower affinity E2 scFv-based CARs exhibited potent cytotoxicity against amph-FITC-tagged MSTO-211H human mesothelioma tumor cells, irrespective of the use of CD28 or 4-1BB costimulatory domains (FIG. 15A). Thus, human CAR T cells also effectively recognized amph-FITC-coated cancer cells.

In most syngeneic murine tumor treatment studies, intratumoral amph-FITC alongside subcutaneously administered amph-FITC (with adjuvant) was administered was efficiently taken up in draining lymph nodes as a vaccine booster. However, because lymphatic vessels and peripheral lymph nodes are defective in the immunodeficient NOD.Cg-PrkdcscidIL2rgtm1Wj1/SzJ (NSG) mice used for human CAR T cell evaluation (see, e.g., Puchalapalli, M. et al. PLoS ONE 11, 1-15 (2016)). FITC-specific CAR T cells were stimulated through intratumoral amph-FITC. To test the in vivo activity of CAR T cells responding to amph-FITC-coated tumors, MSTO-211H were inoculated tumors in NSG mice and adoptively transferred luciferase-expressing 4m5.3 or E2 CAR T cells with CD28 or 4-1BB costimulatory domains 7 days later, and then treated animals with repeated doses of intratumoral amph-FITC (FIG. 6B). In contrast to murine CAR T cells, where transfer of a pure CD8⁺ CAR T cell population had superior therapeutic efficacy to a mixed population of CD4⁺ and CD8⁺ CAR T cells, human CAR T cells were found to proliferate more robustly as a mixed CD4⁺ and CD8⁺ population following amph-FITC administration (FIG. 15B). Accordingly, a mixed CD4/CD8 population was utilized for human T cell experiments. Analysis of the CAR T cells post adoptive transfer showed that these human CAR T cells were comprised of a mixture of Tₑₘ/Tₑₘᵣₐ phenotypes (FIG. 15C-15D).

By treating tumors as shown in FIG. 6B, all of the CAR T cells showed some level of accumulation in tumors between day 13 and day 25, but E2-h28z and E2-hBBz CAR T cells showed the most accumulation at the injected tumor sites (FIG. 6C-6D). CAR T cells in the spleen on day 40 were quantified and showed persistence of all of the CAR T cells systemically, with the exception of the 4m5.3-h28z CAR T cells infused as a pure CD8⁺ population (FIG. 15E). Due to its expression level, in vitro tumor cytotoxicity, and robust accumulation in tumors, the E2-hBBz CAR construct was utilized for human T cell therapy studies. In combination with intratumoral amph-FITC, E2-hBBz CAR T cells led to complete tumor rejection in about 20% of mice and tumor growth was halted in another 60% of the cohort (FIG. 6E). No overt toxicities in terms of animal weight loss or alterations in behavior were noted during treatment until late time points when both treated and untreated groups began showing symptoms of graft vs. host disease, a known limitation of the NSG mouse model. Notably, the 13.3-fold relative expansion of CAR T cells induced by amph-FITC treatment (FIG. 15F) exacerbated GVHD in this group, leading to early death of a number of the animals that were responding to treatment (FIG. 6E). Amph-FITC injection appeared as a promising strategy to redirect human CAR T cells against solid tumors.

What is claimed is:

1. A method of treating a tumor, comprising introducing a membrane-inserting amphiphilic ligand into a tumor of a subject in need of treatment followed by administering an engineered immune cell expressing a CAR—that specifically binds to the amphiphilic ligand.

2. The method of claim 1, wherein the membrane-inserting amphiphilic ligand is a fluorescein isothiocyanate lipid amphiphile ligand.

3. The method of claim 1, wherein the engineered immune cell expressing a CAR that specifically binds to the amphiphilic ligand is a fluorescein isothiocyanate (FITC) lipid amphiphile-specific engineered immune cell.

4. The method of claim 1, wherein the membrane-inserting amphiphilic ligand comprises 1,2-distearoyl-sn-glycero-3-phosphoethanolamine-PEG-FITC.

5. The method of claim 1, wherein the membrane-inserting amphiphilic ligand is introduced into the tumor by intratumoral injection.

6. The method of claim 1, wherein administering the engineered immune cell expressing a CAR that specifically binds to the amphiphilic ligand comprises systemic infusion of the engineered immune cell into the subject.

7. The method of claim 1, further comprising introducing the membrane-inserting amphiphilic ligand into dendritic cells in lymph nodes of the subject by subcutaneous injection.

8. The method of claim 1, wherein the tumor of a subject in need of treatment is a solid tumor.

9. The method of claim 1, wherein the membrane-inserting amphiphilic ligand comprises a therapeutic compound conjugated to an amphiphilic poly(ethylene glycol)-lipid.

10. The method of claim 5, wherein the frequency of intratumoral injection is equal to or less than about once every 6 days.

11. The method of claim 1, wherein the engineered immune cell expressing a CAR comprises a CD28 costimulatory domain.

12. An engineered immune cell expressing a CAR that specifically binds to a membrane-inserted amphiphilic ligand of a tumor cell.

13. The cell of claim 12, wherein the membrane-inserting amphiphilic ligand is a fluorescein isothiocyanate lipid amphiphile ligand.

14. The cell of claim 12, comprising a chimeric antigen receptor that recognizes the amphiphilic ligand.

15. The cell of claim 12, wherein the chimeric antigen receptor comprises amphiphilic-ligand specific scFV.

16. The cell of claim 12, further comprising a CD28 costimulatory domain.

17. The cell of claim 12, wherein the fluorescein isothiocyanate lipid amphiphile ligand is 1,2-distearoyl-sn-glycero-3-phosphoethanolamine-PEG-FITC.
